SEQUENCE LISTING (11 sequences)

SEQ ID NO: 1 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETERISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - protein, 96 aa, Artificial Sequence
  Synthetic amino acid sequence COP-5
LYVDFSDVGWDDWIVAPPGYQAFYCHGECPFPLADHFNSTNHAVVQTLVNSVNSKIPKACCVPTELSAISMLYLDENEKVVLKYNQEMVVEGCGCR

SEQ ID NO: 3 - protein, 96 aa, Artificial Sequence
  Synthetic amino acid sequence COP-7
LYVDFSDVGWNDWIVAPPGYHAFYCHGECPFPLADHLNSTNHAVVQTLVNSVNSKIPKACCVPTELSAISMLYLDENEKVVLKYNQEMVVEGCGCR

SEQ ID NO: 4 - protein, 102 aa, Artificial Sequence
  Synthethic amino acid sequence OP-X
CXXHELYVXFXDLGWXDWXIAPXGYXAYYCEGECXFPLXSXMNATNHAIXQXLVHXXXPXXVPKXCCAPTXLXAXSVLYXDXSXNVXLXKXRNMVVXACGCH

SEQ ID NO: 5 - protein, 97 aa, Artificial Sequence
  Synthetic amino acid sequence Generic Sequence 7
LXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 6 - protein, 102 aa, Artificial Sequence
  Synthetic amino acid sequence Generic Sequence 8
CXXXXLXXXFXXXGWXXXXXXPXXXXAXYCXGXCXXPXXXXXXXXNHAXXXXXXXXXXXXXXXXXCCXPXXXXXXXXLXXXXXXXVXLXXXXXMXVXXCXCX

SEQ ID NO: 7 - protein, 97 aa, Artificial Sequence
  Synthetic amino acid sequence Generic Sequence 9
XXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 8 - protein, 102 aa, Artificial Sequence
  Synthetic amino acid sequence Generic Sequence 10
CXXXXXXXXXXXXXXXXXXXXPXXXXXXXCXGXCXXXXXXXXXXXXXXXXXXXXXXXXXXXXXXXXCXPXXXXXXXXLXXXXXXXXXXXXXXXXXXXXCXCX

SEQ ID NO: 9 - protein, 5 aa, Artificial Sequence
  Consensus Sequence
CXXXX

SEQ ID NO: 10 - DNA, 1822 bp, Homo sapiens
  CDS (49)..(1341)
ggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatgaaaaaaaaaaaaaaaaaa

SEQ ID NO: 11 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

(19) United States
(12) Patent Application Publication
(10) Pub. No.: US 2006/0194726 A1
(43) Pub. Date: Aug. 31, 2006

(54) Methods of treating cartilage defects

(76) Inventors: David C. Rueger, Southborough, MA (US); Robyn Kildey, Sidney (AU)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/286,364

(22) Filed: Nov. 23, 2005

Related U.S. Application Data
(63) Continuation-in-part of application No. PCT/US05/18020, filed on May 24, 2005.
(60) Provisional application No. 60/574,423, filed on May 25, 2004.

(51) Int. Cl. A61K 38/18

(52) U.S. Cl. 514/12

(57) ABSTRACT

The present invention provides methods of repairing and regenerating cartilage tissue by administering into the cartilage or the area surrounding the cartilage a composition comprising a therapeutically effective amount of a morphogenic protein.

### FIELD OF THE INVENTION

The present invention relates to orthopaedic tissue repair. More particularly, it relates to methods of repairing or regenerating cartilage.

### BACKGROUND OF THE INVENTION

Cartilage repair and regeneration is one of the major obstacles in current orthopedics. The importance is enormous because cartilage injury and degenerative disorders such as osteoarthritis, intervertebral disc degeneration and meniscal tears are a major cause of disability among the adult population in the United States.

Cartilage is connective tissue composed of chondrocytes embedded in an extracellular matrix of collagen fibers, proteoglycans, and other non-collagenous proteins. There are two forms of cartilage—articular and non-articular. Articular cartilage is a thin layer of connective tissue, which covers the ends of bones in joints. Non-articular cartilage includes fibrocartilage and elastic cartilage and includes intervertebral discs, meniscus, trachea, larynx, nose, ear and ribs.

The function of cartilage is to cushion load bearing, resist wear, and allow for almost frictionless movement of joints. Defects in cartilage tissue, often caused by trauma, abnormal wear or disease, can lead to pain and stiffness, and if left untreated, may progress and ultimately require replacement of the entire joint. For example, articular cartilage defects often lead to early degradation of the articular surface and may eventually result in osteochondral defects, osteoarthritis or both.

Osteoarthritis is considered a process of attempted, but gradually failing, repair of damaged cartilage extracellular matrix, as the balance between synthesis and breakdown of matrix components is disturbed and shifted toward catabolism.

The ability of cartilage tissue to regenerate on its own is severely limited due to its avascular nature. Repair of osteochondral defects, which involves both the cartilage tissue and the underlying bone, occurs to a limited extent promoted by the presence of both stem cells and growth and differentiation factors brought into the defect by the blood and/or marrow. In animal studies, these defects undergo some repair with formation of a new layer of bone and cartilage, but the macromolecular organization and the biochemical characteristics of the cartilage matrix are imperfect. Type I collagen, rather than Type II collagen, and proteoglycans that are not cartilage specific, such as dermatan sulfate containing proteoglycans, make up the repair tissue and result in fibrillations and degenerative changes over time. And, repair of cartilage defects that do not penetrate into the subchondral bone does not occur, even to a limited extent.

Moreover, surgical treatment of cartilage defects is complex and has been demonstrated to have only limited success. For example, articular cartilage defects are treated with an arthroscopic approach where loose bodies are debrided and transition areas are smoothed. However, this method alone frequently does not provide long lasting relief of the symptoms. Knee replacements often require resecting significant amounts of bone and often require multiple surgeries.

The meniscus is a small horseshoe shaped tissue located between the bone ends inside the knee joint, which acts as a shock absorber. There are two menisci in each knee on either side of the knee. They are usually strong in young people and with age become more brittle and tear more easily. Tears are extremely common with anterior cruciate ligament (ACL) injuries. Meniscal fibrocartilage, like articular hyaline cartilage, has a limited capacity to heal, particularly in the middle and inner avascular regions. The current treatment for small tears is to leave them alone if they do not cause much trouble. Surgical options for treating meniscal tears depend on a number of factors including the nature and extent of the injury and most importantly, its location. Tears in the vascularized region, which is integrated with the highly vascularized synovium have been successfully repaired by suturing. Partial or total meniscectomy is the normal surgical treatment for symptomatic tears within the avascular two thirds of the meniscus. Tears in the latter meniscus regions are the most common types seen clinically. Irrespective of whether open, arthroscopic, total or partial meniscectomy are employed, osteoarthritis is a frequent sequela in these patients within a few years post surgery. Therefore, the common form of repair is to only partially remove the torn bits and to repair the cartilage by stapling it. Unfortunately, the healing process following this procedure is slow. Moreover, if the repair is not successful, then the entire torn meniscus must subsequently be removed.

The major cause of persistent and often debilitating back pain is intervertebral disc (IVD) degeneration. As discs degenerate, they cause the adjoining vertebrae to become compressed, often resulting in severe pain.

The IVD as a syndesmosis provides articulation between adjoining vertebral bodies and acts as a weight bearing cushion which dissipates axially applied spinal loads. These biomechanical functions are made possible by the unique structure of the IVD which is composed of an outer collagen-rich annulus fibrosus surrounding a central hydrated proteoglycan rich gelatinous nucleus pulposus. Superior and inferior cartilaginous endplates, thin layers of hyaline-like cartilage covers the interfaces of the vertebral bodies within the disc.

Lumbar disc degeneration represents a substantial social and economic burden to the community which is manifest principally as low back pain (LBP). It is estimated that as much as 80% of the population experience at least one significant episode of LBP during life, and approximately 2.5% of the working population will take some sick leave during the year as a result of LBP. The direct costs of LBP in modern Western countries has been estimated at $9 billion, most of which is spent on consulting general practitioners, physical therapists and other conservative practitioners (Williams D A et al., (1998) Health care and indemnity costs across the natural history of disability in occupational low back pain, Spine 23:2329-36). Total indirect expenditure, including surgical management may be ten times higher (Maetzel and Li (2002) The economic burden of low back pain: a review of studies published between 1996 and 2001, Best Prac Res Clin Rheumatol 16:23-30; Walker et al., (2003) The economic burden, Proceedings of the Spine Society of Australia Annual Scientific Meeting, Canberra, Australia).

Disc degeneration is a natural phenomenon that occurs, in most instances, from the time of skeletal maturity (Vernon-Roberts (1992) Age-related and degenerative pathology of intervertebral discs and apophyseal joints, In: The lumbar spine and back pain. Fourth edition, Jayson M I V, Ed. Churchill Livingstone, Edinburgh, Chapter 2, 17-41). It is consistent with advancing age but in many cases is also associated with pain, particularly in the lumbar spine, and restricted mobility. Symptoms of LBP often resolve spontaneously over time as patients modify their lifestyles to accommodate restricted mobility. In many cases however, it remains a significant factor that requires surgical intervention. The traditional “gold standard” surgical treatment for chronic LBP has been spinal fusion to immobilize the one or more painful level. Fusion is expensive because it requires prolonged hospitalization and specialist surgical expertise, and although most of these patients will experience short-term pain relief there is evidence now that fusion does not provide the best outcome. Long-term studies suggest that spinal fusion actually promotes degeneration at levels adjacent to the fusion site (Lee (1988) Accelerated degeneration of the segment adjacent to a lumbar fusion, Spine 13:375-7.). In the same way that artificial prostheses were developed 50 years ago to restore function to arthritic and fractured hips and knees, prostheses are now being developed with the aim of restoring full mechanical function to discs that have become painful and arthritic due to chronic degeneration (Szpaalski et al (2002) V Spine arthroplasty: a historical review, Eur Spine J 11 :S65-S84). It is however too early to know if any of the myriad models undergoing trials will provide long-term benefit.

A class of proteins have now been identified that are competent to act as true bone and cartilage tissue morphogens, able, on their own, to induce the proliferation and differentiation of progenitor cells into functional bone, cartilage, tendon, and/or ligamentous tissue. These proteins, referred to herein as “osteogenic proteins” or “morphogenic proteins” or “morphogens,” includes members of the family of bone morphogenetic proteins (BMPs) which were initially identified by their ability to induce ectopic, endochondral bone morphogenesis. The osteogenic proteins generally are classified in the art as a subgroup of the TGF-β superfamily of growth factors (Hogan (1996) Genes & Development 10:1580-1594). Members of the morphogen family of proteins include the mammalian osteogenic protein-1 (OP-1, also known as BMP-7, and the Drosophila homolog 60A), osteogenic protein-2 (OP-2, also known as BMP-8), osteogenic protein-3 (OP-3), BMP-2 (also known as BMP-2A or CBMP-2A, and the Drosophila homolog DPP), BMP-3, BMP-4 (also known as BMP-2B or CBMP-2B), BMP-5, BMP-6 and its murine homolog Vgr-1, BMP-9, BMP-10, BMP-11, BMP-12, GDF3 (also known as Vgr2), GDF8, GDF9, GDF10, GDF11, GDF12, BMP-13, BMP-14, BMP-15, BMP-16, BMP-17, BMP-18, GDF-5 (also known as CDMP-1 or MP52), GDF-6 (also known as CDMP-2), GDF-7 (also known as CDMP-3), the Xenopus homolog Vg1 and NODAL, UNIVIN, SCREW, ADMP, and NEURAL. Members of this family encode secreted polypeptide chains sharing common structural features, including processing from a precursor “pro-form” to yield a mature polypeptide chain competent to dimerize, and containing a carboxy terminal active domain of approximately 97-106 amino acids. All members share a conserved pattern of cysteines in this domain and the active form of these proteins can be either a disulfide-bonded homodimer of a single family member, or a heterodimer of two different members (see, e.g., Massague (1990) Annu. Rev. Cell Biol. 6:597; Sampath, et al. (1990) J. Biol. Chem. 265:13198). See also, U.S. Pat. No. 5,011,691; U.S. Pat. No. 5,266,683, Ozkaynak et al. (1990) EMBO J. 9: 2085-2093, Wharton et al. (1991) PNAS 88:9214-9218), (Ozkaynak (1992) J. Biol. Chem. 267:25220-25227 and U.S. Pat. No. 5,266,683); (Celeste et al. (1991) PNAS 87:9843-9847); (Lyons et al. (1989) PNAS 86:4554-4558). These disclosures describe the amino acid and DNA sequences, as well as the chemical and physical characteristics of these osteogenic proteins. See also Wozney et al. (1988) Science 242:1528-1534); BMP 9 (WO93/00432, published Jan. 7, 1993); DPP (Padgett et al. (1987) Nature 325:81-84; and Vg-1 (Weeks (1987) Cell 51:861-867).

The currently preferred methods of repairing cartilage defects include debridement, microfracture, autologous cell transplantation, mosaicplasty and joint replacement. However, none of these methods, result in actual repair and replacement of cartilage tissue. These methods result in imperfect repair tissue with scar-like characteristics.

Therefore, there remains a need for compositions and methods for repairing and regenerating cartilage defects which overcome the problems associated with the currently available methods and compositions.

### SUMMARY OF THE INVENTION

The present invention provides methods of repairing and regenerating cartilage tissue by administering into the cartilage or into the area surrounding the cartilage a composition comprising a morphogenic protein. In some embodiments, the present invention provides a method of repairing a cartilage defect in a patient comprising the step of administering into the cartilage or into the area surrounding the cartilage a composition comprising a therapeutically effective amount of a morphogenic protein.

The present invention also provides a method of regenerating or producing cartilage in a patient comprising the step of administering into the cartilage or into the area surrounding the cartilage a composition comprising a therapeutically effective amount of a morphogenic protein. In some embodiments, the invention provides a method of regenerating cartilage in a patient comprising the step of administering into the cartilage or into the area surrounding the cartilage a composition comprising a therapeutically effective amount of a morphogenic protein. In other embodiments, the present invention provides a method of producing cartilage in a patient comprising the step of administering into the cartilage or into the area surrounding the cartilage a composition comprising a therapeutically effective amount of a morphogenic protein.

The present invention also provides a method of promoting cartilage growth or accelerating cartilage formation in a patient comprising the step of administering into the cartilage or into the area surrounding the cartilage a composition comprising a therapeutically effective amount of a morphogenic protein. In some embodiments, the invention provides a method of promoting cartilage growth in a patient comprising the step of administering into the cartilage or into the area surrounding the cartilage a composition comprising a therapeutically effective amount of a morphogenic protein. In other embodiments, the invention provides a method of accelerating cartilage formation in a patient comprising the step of administering into the cartilage or into the area surrounding the cartilage a composition comprising a therapeutically effective amount of a morphogenic protein.

The present invention also provides a method of preventing cartilage degradation or treating cartilage tissue injury or degenerative disease or disorder in a patient comprising the step of administering into the cartilage or into the area surrounding the cartilage a composition comprising a therapeutically effective amount of a morphogenic protein. In some embodiments, the invention provides a method of preventing cartilage degradation in a patient comprising the step of administering into the cartilage or into the area surrounding the cartilage a composition comprising a therapeutically effective amount of a morphogenic protein. In other embodiments, the invention provides a method of treating cartilage tissue injury or degenerative disease or disorder. In some embodiments the tissue injury or degenerative disease includes but is not limited to osteoarthritis, meniscus tears, ACL injury and disc degeneration.

In some embodiments, the cartilage is articular cartilage. In other embodiments, the cartilage is non-articular cartilage. In some embodiments, the non-articular cartilage is a meniscus or an intervertebral disc.

In some embodiments, the composition is administered into the cartilage. In some embodiments, the composition is administered into a meniscus or an intervertebral disc. In some embodiments, the composition is administered into the areas surrounding the cartilage. In some embodiments, the area surrounding the cartilage is synovial fluid.

In some embodiments, the morphogenic protein in the composition used in the methods of this invention includes but is not limited to OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, fragments thereof, and amino acid sequence variants thereof. In other embodiments, the morphogenic protein comprises an amino acid sequence having at least 70% homology with the C-terminal 102-106 amino acids, including the conserved seven cysteine domain, of human OP-1, said morphogenic protein being capable of inducing repair of the cartilage defect. In a preferred embodiment, the morphogenic protein is selected from OP-1, BMP-5, BMP-6, GDF-5, GDF-6, GDF-7, CDMP-1, CDMP-2 and CDMP-3. In a more preferred embodiment, the morphogenic protein is OP-1.

In some embodiments, the morphogenic protein composition used in the methods of this invention includes but is not limited to a gel, a putty, a paste or an aqueous solution. In some embodiments, the morphogenic protein composition is formulated as a sustained release formulation or as a delayed clearance formulation (i.e., a formulation whereby the clearance of the morphogenic protein is delayed relative to its normal clearance). In some embodiments, the morphogenic protein composition is formulated as an injectable formulation.

### BRIEF DESCRIPTION OF THE DRAWINGS

FIG. 1 is a schematic of a joint showing the site of the bilateral impact injuries.

FIG. 2 is a graph showing the number of leucocytes in the synovial fluid for OP-1-treated and control animals.

FIGS. 3A and 3B are histological sections of control and OP-1-treated joints.

FIG. 4 is a graph showing the sGAG levels in medial femoral condyle cartilage for OP-1-treated and control animals.

FIG. 5 shows representative results of control and OP-1-treated sheep in the osteoarthritis model. Control sheep were treated with collagen alone. OP-1-treated sheep received 350 μg OP-1 putty at the time of surgery and a second dose was injected into the joint space 1 week later.

FIG. 6 is a histological section of hole 6 weeks after treatment with OP-1 putty.

FIG. 7 is a histological section of hole 6 week control defect.

FIG. 8 is a histological section of hole 12 week control defect.

FIG. 9 is a histological section of hole 12 weeks after treatment with OP-1 putty.

FIG. 10 is a histological section of a meniscal tear defect 6 weeks after treatment with OP-1 putty.

FIG. 11 depicts the zonal dissection scheme to separate the disc into annulus fibrosus (AF) quadrants and the nucleus pulposus (NP) and the location and extent of the anterolateral annular lesion in quadrant 1 in horizontal and vertical sections through lumbar ovine intervertebral discs. The location of the AF lesion in horizontal sections 3 and 6 months post surgery is well illustrated on the left hand side of this figure. Vertical histological sections through the intervertebral disc and adjacent vertebral body and superior and inferior vertebral growth plates also demonstrates the focal nature of the AF lesion (arrow) associated with changes in collagen in the outer AF (Masson trichrome) and collagenous organization (picrosirius red) and focal depletion of proteoglycan (toluidine blue) in the lesion site which penetrates approximately 4 mm into the disc (right hand side of figure).

FIG. 12 depicts flexion-extension ROM plots for intact and injured (anterior annular lesion) sheep functional spinal unit (FSU).

FIG. 13 depicts the amino acid sequence of human OP-1.

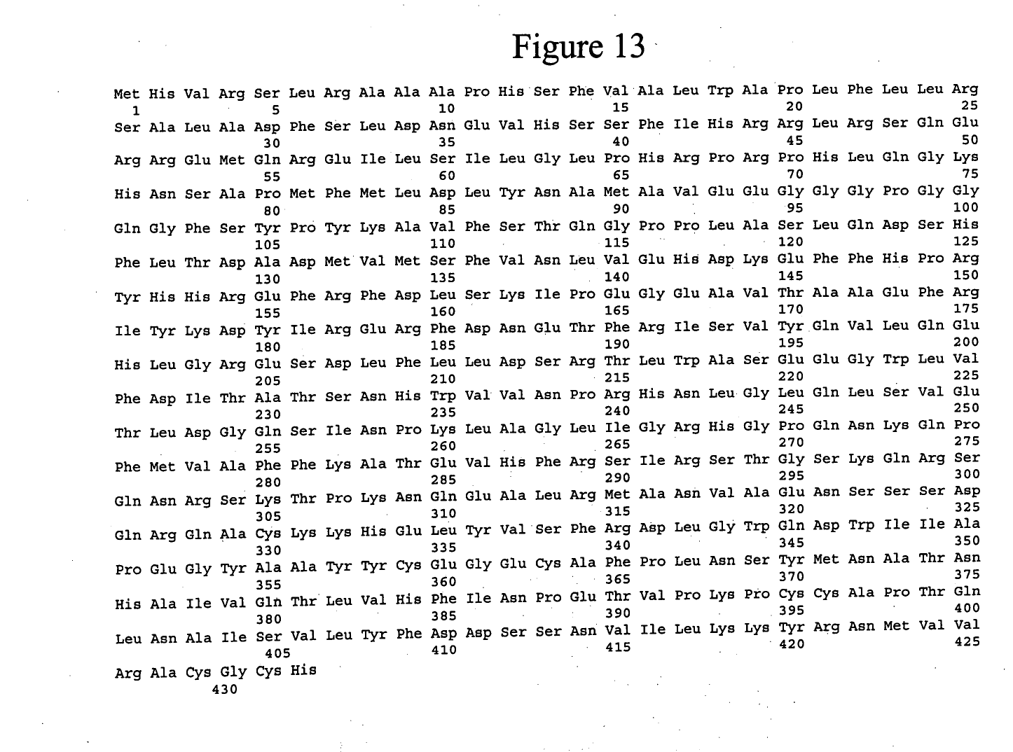

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

In order to further define the invention, the following terms and definitions are provided herein.

The term “cartilage” refers to a type of connective tissue that contains chondrocytes or chondrocyte-like cells (having many, but not all characteristics of chondrocytes) and intercellular material (e.g., Types I, II, IX and XI collagen), proteoglycans (e.g., chondroitin sulfate, keratan sulfate, and dermatan sulfate proteoglycans) and other proteins. Cartilage includes articular and non-articular cartilage.

“Articular cartilage,” also referred to as hyaline cartilage, refers to an avascular, non-mineralized connective tissue, which covers the articulating surfaces of bones in joints and serves as a friction reducing interface between two opposing bone surfaces. Articular cartilage allows movement in joints without direct bone-to-bone contact. Articular cartilage has no tendency to ossification. The cartilage surface appears smooth and pearly macroscopically, and is finely granular under high power magnification. Articular cartilage derives nutrients partly from the vessels of the neighboring synovial membrane and partly from the vessels of the bone it covers. Articular cartilage is associated with the presence of Type II and Type IX collagen and various well-characterized proteoglycans, and with the absence of Type X collagen, which is associated with endochondral bone formation. For a detailed description of articular cartilage microstructure, see, for example, Aydelotte and Kuettner, Conn. Tiss. Res., 18, p. 205 (1988); Zanetti et al., J. Cell Biol., 101, p. 53 (1985); and Poole et al., J. Anat., 138, p. 13 (1984).

“Non-articular cartilage” refers to cartilage that does not cover articulating surfaces and includes fibrocartilage (including interarticular fibrocartilage, fibrocartilaginous disc, connecting fibrocartilage and circumferential fibrocartilage) and elastic cartilage. In fibrocartilage, the micropolysaccharide network is interlaced with prominent collagen bundles, and the chondrocytes are more widely scattered than in hyaline or articular cartilage. Interarticular fibrocartilage is found in joints which are exposed to concussion and subject to frequent movement, e.g., the meniscus of the knee. Examples of such joints include but are not limited to the temporo-mandibular, sterno-clavicular, acromio-clavicular, wrist and knee joints. Secondary cartilaginous joints are formed by discs of fibrocartilage. Such fibrocartilaginous discs, which adhere closely to both of the opposed surfaces, are composed of concentric rings of fibrous tissue, with cartilaginous laminae interposed. An example of such fibrocartilaginous disc is the intervertebral disc of the spine. Connecting fibrocartilage is interposed between the bony surfaces of those joints, which allow for slight mobility as between the bodies of the vertebrae and between the pubic bones. Circumferential fibrocartilage surrounds the margin of some of the articular cavities, such as the cotyloid cavity of the hip and the glenoid cavity of the shoulder.

Elastic cartilage contains fibers of collagen that are histologically similar to elastin fibers. Such cartilage is found in the auricle of the external ear, the eustachian tubes, the cornicula laryngis and the epiglottis. As with all cartilage, elastic cartilage also contains chondrocytes and a matrix, the latter being pervaded in every direction, by a network of yellow elastic fibers, branching and anastomosing in all directions except immediately around each cell, where there is a variable amount of non-fibrillated, hyaline, intercellular substance.

The term “synovial fluid” refers to a thin, lubricating substance within the synovial cavity that reduces friction within the joint.

The term “defect” or “defect site”, refers to a disruption of chondral or osteochondral tissue. A defect can assume the configuration of a “void”, which is understood to mean a three-dimensional defect such as, for example, a gap, cavity, hole or other substantial disruption in the structural integrity of chondral or osteochondral tissue. A defect can also be a detachment of the cartilage from its point of attachment to the bone or ligaments. In certain embodiments, the defect is such that it is incapable of endogenous or spontaneous repair. A defect can be the result of accident, disease, and/or surgical manipulation. For example, cartilage defects may be the result of trauma to a joint such as a displacement of torn meniscus tissue into the joint. Cartilage defects may be also be the result of degenerative joint diseases such as osteoarthritis.

The term “repair” refers to new cartilage formation which is sufficient to at least partially fill the void or structural discontinuity at the defect site. Repair does not, however, mean, or otherwise necessitate, a process of complete healing or a treatment, which is 100% effective at restoring a defect to its pre-defect physiological/structural/mechanical state.

The term “therapeutically effective amount” refers to an amount effective to repair, regenerate, promote, accelerate, prevent degradation, or form cartilage tissue.

The term “patient” refers to an animal including a mammal (e.g., a human).

The term “pharmaceutically acceptable carrier of adjuvant” refers to a non-toxic carrier or adjuvant that may be administered to a patient, together with a morphogenic protein of this invention, and which does not destroy the pharmacological activity thereof.

The term “morphogenic protein” refers to a protein having morphogenic activity. Preferably a morphogenic protein of this invention comprises at least one polypeptide belonging to the BMP protein family. Morphogenic proteins include osteogenic proteins. Morphogenic proteins may be capable of inducing progenitor cells to proliferate and/or to initiate differentiation pathways that lead to cartilage, bone, tendon, ligament or other types of tissue formation depending on local environmental cues, and thus morphogenic proteins may behave differently in different surroundings. For example, a morphogenic protein may induce bone tissue at one treatment site and cartilage tissue at a different treatment site.

The term “bone morphogenic protein (BMP)” refers to a protein belonging to the BMP family of the TGF-β superfamily of proteins (BMP family) based on DNA and amino acid sequence homology. A protein belongs to the BMP family according to this invention when it has at least 50% amino acid sequence identity with at least one known BMP family member within the conserved C-terminal cysteine-rich domain, which characterizes the BMP protein family. Preferably, the protein has at least 70% amino acid sequence identity with at least one known BMP family member within the conserved C-terminal cysteine rich domain. Members of the BMP family may have less than 50% DNA or amino acid sequence identity overall. Osteogenic protein as defined herein also is competent to induce articular cartilage formation at an appropriate in vivo avascular locus.

The term “amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. Certain particularly preferred morphogenic polypeptides share at least 60%, and preferably 70% amino acid sequence identity with the C-terminal 102-106 amino acids, defining the conserved seven-cysteine domain of human OP-1 and related proteins.

Amino acid sequence homology can be determined by methods well known in the art. For instance, to determine the percent homology of a candidate amino acid sequence to the sequence of the seven-cysteine domain, the two sequences are first aligned. The alignment can be made with, e.g., the dynamic programming algorithm described in Needleman et al., J. Mol. Biol., 48, pp. 443 (1970), and the Align Program, a commercial software package produced by DNAstar, Inc. The teachings by both sources are incorporated by reference herein. An initial alignment can be refined by comparison to a multi-sequence alignment of a family of related proteins. Once the alignment is made and refined, a percent homology score is calculated. The aligned amino acid residues of the two sequences are compared sequentially for their similarity to each other. Similarity factors include similar size, shape and electrical charge. One particularly preferred method of determining amino acid similarities is the PAM250 matrix described in Dayhoff et al., Atlas of Protein Sequence and Structure, 5, pp. 345-352 (1978 & Supp.), which is incorporated herein by reference. A similarity score is first calculated as the sum of the aligned pair wise amino acid similarity scores. Insertions and deletions are ignored for the purposes of percent homology and identity. Accordingly, gap penalties are not used in this calculation. The raw score is then normalized by dividing it by the geometric mean of the scores of the candidate sequence and the seven-cysteine domain. The geometric mean is the square root of the product of these scores. The normalized raw score is the percent homology.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., supra. Examples of conservative substitutions are substitutions within the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative variant” or “conservative variation” also includes the use of a substituting amino acid residue in place of an amino acid residue in a given parent amino acid sequence, where antibodies specific for the parent sequence are also specific for, i.e., “cross-react” or “immuno-react” with, the resulting substituted polypeptide sequence.

The term “osteogenic protein (OP)” refers to a morphogenic protein that is capable of inducing a progenitor cell to form cartilage and/or bone. The bone may be intramembranous bone or endochondral bone. Most osteogenic proteins are members of the BMP protein family and are thus also BMPs. As described elsewhere herein, the class of proteins is typified by human osteogenic protein (hOP-1). Other osteogenic proteins useful in the practice of the invention include osteogenically active forms of OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr-1, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, UNIVIN, NODAL, SCREW, ADMP or NEURAL, and amino acid sequence variants thereof. Osteogenic proteins suitable for use with applicants' invention can be identified by means of routine experimentation using the art-recognized bioassay described by Reddi and Sampath (Sampath et al., Proc. Natl. Acad. Sci., 84, pp. 7109-13, incorporated herein by reference).

Methods and Compositions For Cartilage Growth and Repair

The morphogenic compositions of this invention may be used for cartilage repair (e.g., at a joint, meniscus or intervertebral disc). The morphogenic compositions comprising a morphogenic protein disclosed herein will permit the physician to treat a variety of tissue injuries, tissue degenerative or disease conditions and disorders that can be ameliorated or remedied by localized, stimulated tissue regeneration or repair.

The invention provides methods and compositions for treating cartilage tissue injuries and cartilage degenerative diseases or disorders including but not limited to osteoarthritis, meniscus tears, ACL injuries and disc degeneration.

In some embodiments, the invention provides methods and compositions for repairing or regenerating cartilage in a patient. The invention also provides methods and compositions for producing cartilage, promoting cartilage growth accelerating cartilage formation and preventing cartilage degradation in a patient.

In some embodiments, the methods of the present invention comprise the step of administering into the cartilage a composition comprising a therapeutically effective amount of a morphogenic protein. This method involves the administration of the morphogenic composition directly into the cartilage tissue (e.g., an injection into the cartilage tissue). For example, the morphogenic protein composition may be injected into a meniscus or an intervertebral disc. In some embodiments, the methods of the present invention comprise the step of administering into the synovial fluid surrounding the cartilage a composition comprising a therapeutically effective amount of a morphogenic protein. In some embodiments, the cartilage is articular cartilage. In other embodiments, the cartilage is non-articular cartilage. In some embodiments, the non-articular cartilage includes but is not limited to intervertebral disc, interarticular meniscus, trachea, ear, nose, rib and larynx. In a preferred embodiment the non-articular cartilage is an intervertebral disc. In another preferred embodiment, the non-articular cartilage is a meniscus. In some embodiments, the area surrounding the cartilage is synovial fluid.

In some embodiments, the morphogenic protein in the composition used in the methods of the present invention includes but is not limited to OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and amino acid sequence variants thereof. In other embodiments, the morphogenic protein comprises an amino acid sequence having at least 70% homology with the C-terminal 102-106 amino acids, including the conserved seven cysteine domain, of human OP-1, said morphogenic protein being capable of inducing repair of the cartilage defect. In a preferred embodiment, the morphogenic protein is OP-1, BMP-5, BMP-6, GDF-5, GDF-6, GDF-7, CDMP-1, CDMP-2 or CDMP-3. In a more preferred embodiment, the morphogenic protein is OP-1.

Bone Morphogenic Protein Family

The BMP family, named for its representative bone morphogenic/osteogenic protein family members, belongs to the TGF-β protein superfamily. Of the reported “BMPs” (BMP-1 to BMP-18), isolated primarily based on sequence homology, all but BMP-1 remain classified as members of the BMP family of morphogenic proteins (Ozkaynak et al., EMBO J., 9, pp. 2085-93 (1990)).

The BMP family includes other structurally-related members which are morphogenic proteins, including the drosophila decapentaplegic gene complex (DPP) products, the Vg1 product of Xenopus laevis and its murine homolog, Vgr-1 (see, e.g., Massagué, Annu. Rev. Cell Biol., 6, pp. 597-641 (1990), incorporated herein by reference).

The C-terminal domains of BMP-3, BMP-5, BMP-6, and OP-1 (BMP-7) are about 60% identical to that of BMP-2, and the C-terminal domains of BMP-6 and OP-1 are 87% identical. BMP-6 is likely the human homolog of the murine Vgr-1 (Lyons et al., Proc. Natl. Acad. Sci. U.S.A., 86, pp. 4554-59 (1989)); the two proteins are 92% identical overall at the amino acid sequence level (U.S. Pat. No. 5,459,047, incorporated herein by reference). BMP-6 is 58% identical to the Xenopus Vg-1 product.

Biochemical Structural and Functional Properties of Bone Morphogenic Proteins

The naturally occurring bone morphogens share substantial amino acid sequence homology in their C-terminal regions (domains). Typically, the above-mentioned naturally occurring osteogenic proteins are translated as a precursor, having an N-terminal signal peptide sequence typically less than about 30 residues, followed by a “pro” domain that is cleaved to yield the mature C-terminal domain of approximately 97-106 amino acids. The signal peptide is cleaved rapidly upon translation, at a cleavage site that can be predicted in a given sequence using the method of Von Heijne Nucleic Acids Research, 14, pp. 4683-4691 (1986). The pro domain typically is about three times larger than the fully processed mature C-terminal domain.

Another characteristic of the BMP protein family members is their apparent ability to dimerize. Several bone-derived osteogenic proteins (OPs) and BMPs are found as homo- and heterodimers in their active forms. The ability of OPs and BMPs to form heterodimers may confer additional or altered morphogenic inductive capabilities on morphogenic proteins. Heterodimers may exhibit qualitatively or quantitatively different binding affinities than homodimers for OP and BMP receptor molecules. Altered binding affinities may in turn lead to differential activation of receptors that mediate different signaling pathways, which may ultimately lead to different biological activities or outcomes. Altered binding affinities could also be manifested in a tissue or cell type-specific manner, thereby inducing only particular progenitor cell types to undergo proliferation and/or differentiation.

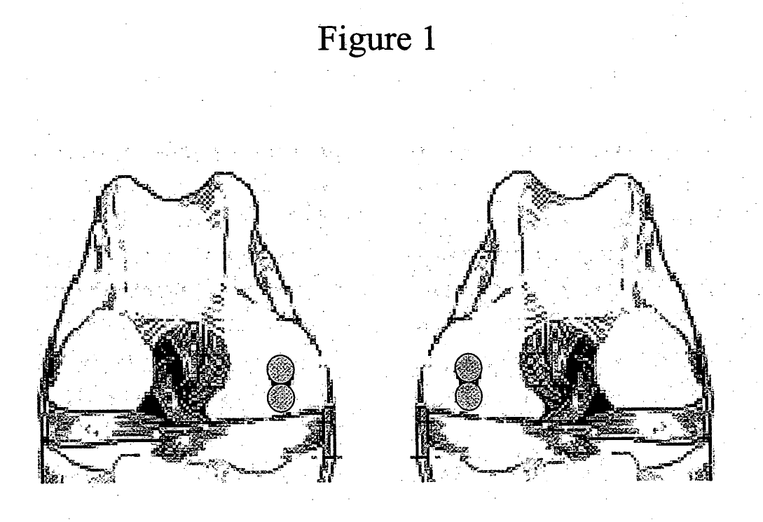

In preferred embodiments, the pair of osteogenic polypeptides have amino acid sequences each comprising a sequence that shares a defined relationship with an amino acid sequence of a reference morphogen. Herein, preferred osteogenic polypeptides share a defined relationship with a sequence present in osteogenically active human OP-1, SEQ ID NO: 1 (See FIG. 1). However, any one or more of the naturally occurring or biosynthetic sequences disclosed herein similarly could be used as a reference sequence. Preferred osteogenic polypeptides share a defined relationship with at least the C-terminal six cysteine domain of human OP-1, residues 335-431 of SEQ ID NO: 1. Preferably, osteogenic polypeptides share a defined relationship with at least the C-terminal seven cysteine domain of human OP-1, residues 330-431 of SEQ ID NO: 1. That is, preferred polypeptides in a dimeric protein with bone morphogenic activity each comprise a sequence that corresponds to a reference sequence or is functionally equivalent thereto.

Functionally equivalent sequences include functionally equivalent arrangements of cysteine residues disposed within the reference sequence, including amino acid insertions or deletions which alter the linear arrangement of these cysteines, but do not materially impair their relationship in the folded structure of the dimeric morphogenic protein, including their ability to form such intra- or inter-chain disulfide bonds as may be necessary for morphogenic activity. Functionally equivalent sequences further include those wherein one or more amino acid residues differs from the corresponding residue of a reference sequence, e.g., the C-terminal seven cysteine domain (also referred to herein as the conserved seven cysteine skeleton) of human OP-1, provided that this difference does not destroy bone morphogenic activity. Accordingly, conservative substitutions of corresponding amino acids in the reference sequence are preferred. Amino acid residues that are conservative substitutions for corresponding residues in a reference sequence are those that are physically or functionally similar to the corresponding reference residues, e.g., that have similar size, shape, electric charge, chemical properties including the ability to form covalent or hydrogen bonds, or the like. Particularly preferred conservative substitutions are those fulfilling the criteria defined for an accepted point mutation in Dayhoff et al., supra, the teachings of which are incorporated by reference herein.

The osteogenic protein OP-1 has been described (see, e.g., Oppermann et al., U.S. Pat. No. 5,354,557, incorporated herein by reference). Natural-sourced osteogenic protein in its mature, native form is a glycosylated dimer typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. In the reduced state, the protein has no detectable osteogenic activity. The unglycosylated protein, which also has osteogenic activity, has an apparent molecular weight of about 27 kDa. When reduced, the 27 kDa protein gives rise to two unglycosylated polypeptides, having molecular weights of about 14 kDa to 16 kDa, capable of inducing endochondral bone formation in a mammal. Osteogenic proteins may include forms having varying glycosylation patterns, varying N-termini, and active truncated or mutated forms of native protein. As described above, particularly useful sequences include those comprising the C-terminal 96 or 102 amino acid sequences of DPP (from Drosophila), Vg1 (from Xenopus), Vgr-1 (from mouse), the OP-1 and OP-2 proteins,(see U.S. Pat. No. 5,011,691 and Oppermann et al., incorporated herein by reference), as well as the proteins referred to as BMP-2, BMP-3, BMP-4 (see WO88/00205, U.S. Pat. No. 5,013,649 and WO91/18098, incorporated herein by reference), BMP-5 and BMP-6 (see WO90/11366, PCT/US90/01630, incorporated herein by reference), BMP-8 and BMP-9.

Preferred morphogenic and osteogenic proteins of this invention comprise at least one polypeptide including, but not limited to OP-1 (BMP-7), OP-2, OP-3, COP-1, COP-3, COP-4, COP-5, COP-7, COP-16, BMP-2, BMP-3, BMP-3b, BMP-4, BMP-5, BMP-6, BMP-9, BMP-10, BMP-11, BMP-12, BMP-13, BMP-14, BMP-15, BMP-16, BMP-17, BMP-18, GDF-1, GDF-2, GDF-3, GDF-5, GDF-6, GDF-7, GDF-8, GDF-9, GDF-10, GDF-11, GDF-12, MP121, CDMP-1, CDMP-2, CDMP-3, dorsalin-1, DPP, Vg-1, Vgr-1, 60A protein, NODAL, UNIVIN, SCREW, ADMP, NEURAL, TGF-β and amino acid sequence variants and homologs thereof, including species homologs, thereof. Preferably, the morphogenic protein comprises at least one polypeptide selected from OP-1 (BMP-7), BMP-2, BMP-4, BMP-5, BMP-6, GDF-5, GDF-6, GDF-7, CDMP-1, CDMP-2 or CDMP-3; more preferably, OP-1 (BMP-7) BMP-5, BMP-6, GDF-5, GDF-6, GDF-7, CDMP-1, CDMP-2 or CDMP-3; and most preferably, OP-1 (BMP-7).

Publications disclosing these sequences, as well as their chemical and physical properties, include: OP-1 and OP-2 (U.S. Pat. No. 5,011,691; U.S. Pat. No. 5,266,683; Ozkaynak et al., EMBO J., 9, pp. 2085-2093 (1990); OP-3 (WO94/10203 (PCT US93/10520)); BMP-2, BMP-3, BMP-4, (WO88/00205; Wozney et al. Science, 242, pp. 1528-1534 (1988)); BMP-5 and BMP-6, (Celeste et al., PNAS, 87, 9843-9847 (1991)); Vgr-1 (Lyons et al., PNAS, 86, pp. 4554-4558 (1989)); DPP (Padgett et al. Nature, 325, pp. 81-84 (1987)); Vg-1 (Weeks, Cell, 51, pp. 861-867 (1987)); BMP-9 (WO95/33830 (PCT/US95/07084); BMP-10 (WO94/26893 (PCT/US94/05290); BMP-11 (WO94/26892 (PCT/US94/05288); BMP-12 (WO95/16035 (PCT/US94/14030); BMP-13 (WO95/16035 (PCT/US94/14030); GDF-1 (WO92/00382 (PCT/US91/04096) and Lee et al. PNAS, 88, pp. 4250-4254 (1991); GDF-8 (WO94/21681 (PCT/US94/03019); GDF-9 (WO94/15966 (PCT/US94/00685); GDF-10 (WO95/10539 (PCT/US94/11440); GDF-1 1 (WO96/01845 (PCT/US95/08543); BMP-15 (WO96/36710 (PCT/US96/06540); MP-121 (WO96/01316 (PCT/EP95/02552); GDF-5 (CDMP-1, MP52) (WO94/15949 (PCT/US94/00657) and W096/14335 (PCT/US94/12814) and WO93/16099 (PCT/EP93/00350)); GDF-6 (CDMP-2, BMP13) (WO95/01801 (PCT/US94/07762) and WO96/14335 and WO95/10635 (PCT/US94/14030)); GDF-7 (CDMP-3, BMP12) (WO95/10802 (PCT/US94/07799) and WO95/10635 (PCT/US94/14030)); BMP-17 and BMP-18 (U.S. Pat. No. 6,027,917). The above publications are incorporated herein by reference.

In another embodiment, useful proteins include biologically active biosynthetic constructs, including novel biosynthetic morphogenic proteins and chimeric proteins designed using sequences from two or more known morphogens.

In another embodiment of this invention, a morphogenic protein may be prepared synthetically to induce tissue formation. Morphogenic proteins prepared synthetically may be native, or may be non-native proteins, i.e., those not otherwise found in nature.

Non-native osteogenic proteins have been synthesized using a series of consensus DNA sequences (U.S. Pat. No. 5,324,819, incorporated herein by reference). These consensus sequences were designed based on partial amino acid sequence data obtained from natural osteogenic products and on their observed homologies with other genes reported in the literature having a presumed or demonstrated developmental function.

Several of the biosynthetic consensus sequences (called consensus osteogenic proteins or “COPs”) have been expressed as fusion proteins in prokaryotes (see, e.g., U.S. Pat. No. 5,011,691, incorporated herein by reference. These include COP-1, COP-3, COP-4, COP-5, COP-7 and COP-16, as well as other proteins known in the art. Purified fusion proteins may be cleaved, refolded, implanted in an established animal model and shown to have bone- and/or cartilage-inducing activity. The currently preferred synthetic osteogenic proteins comprise two synthetic amino acid sequences designated COP-5 (SEQ. ID NO: 2) and COP-7 (SEQ. ID NO: 3).

Oppermann et al., U.S. Pat. Nos. 5,011,691 and 5,324,819, which are incorporated herein by reference, describe the amino acid sequences of COP-5 and COP-7 as shown below:

| | |
|---|---|
| COP5 | LYVDFS-DVGWDDWJVAPPGYQAFYCHGECPFPLAD |
| COP7 | LYVDFS-DVGWNDWJVAPPGYHAFYCHGECPFPLAD |
| COP5 | HFNSTN--H-AVVQTLVNSVNSKI--PKACCVPTELSA |
| COP7 | HLNSTN--H-AVVQTLVNSVNSKI--PKACCVPTELSA |
| COP5 | ISMLYLDENEKVVLKYNQEMVVEGCGCR |
| COP7 | ISMLYLDENEKVVLKYNQEMVVEGCGCR |

In these amino acid sequences, the dashes (-) are used as fillers only to line up comparable sequences in related proteins. Differences between the aligned amino acid sequences are highlighted.

The DNA and amino acid sequences of these and other BMP family members are published and may be used by those of skill in the art to determine whether a newly identified protein belongs to the BMP family. New BMP-related gene products are expected by analogy to possess at least one morphogenic activity and thus classified as a BMP.

In one preferred embodiment of this invention, the morphogenic protein comprises a pair of subunits disulfide bonded to produce a dimeric species, wherein at least one of the subunits comprises a recombinant peptide belonging to the BMP protein family. In another preferred embodiment of this invention, the morphogenic protein comprises a pair of subunits that produce a dimeric species formed through non-covalent interactions, wherein at least one of the subunits comprises a recombinant peptide belonging to the BMP protein family. Non-covalent interactions include Van der Waals, hydrogen bond, hydrophobic and electrostatic interactions. The dimeric species may be a homodimer or heterodimer and is capable of inducing cell proliferation and/or tissue formation.

In certain preferred embodiments, bone morphogenic proteins useful herein include those in which the amino acid sequences comprise a sequence sharing at least 70% amino acid sequence homology or “similarity”, and preferably 75%, 80%, 85%, 90%, 95%, or 98% homology or similarity, with a reference morphogenic protein selected from the foregoing naturally occurring proteins. Preferably, the reference protein is human OP-1, and the reference sequence thereof is the C-terminal seven cysteine domain present in osteogenically active forms of human OP-1, residues 330-431 of SEQ ID NO: 1. In certain embodiments, a polypeptide suspected of being functionally equivalent to a reference morphogen polypeptide is aligned therewith using the method of Needleman, et al., supra, implemented conveniently by computer programs such as the Align program (DNAstar, Inc.). As noted above, internal gaps and amino acid insertions in the candidate sequence are ignored for purposes of calculating the defined relationship, conventionally expressed as a level of amino acid sequence homology or identity, between the candidate and reference sequences. “Amino acid sequence homology” is understood herein to include both amino acid sequence identity and similarity. Homologous sequences share identical and/or similar amino acid residues, where similar residues are conservation substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence. In a currently preferred embodiment, the reference sequence is OP-1. Bone morphogenic proteins useful herein accordingly include allelic, phylogenetic counterpart and other variants of the preferred reference sequence, whether naturally-occurring or biosynthetically produced (e.g., including “muteins” or “mutant proteins”), as well as novel members of the general morphogenic family of proteins, including those set forth and identified above. Certain particularly preferred morphogenic polypeptides share at least 60% amino acid identity with the preferred reference sequence of human OP-1, still more preferably at least 65%, 70%, 75%, 80%, 85%, 90%, 95%, or 98% amino acid identity therewith.

In another embodiment, useful osteogenic proteins include those sharing the conserved seven cysteine domain and sharing at least 70% amino acid sequence homology (similarity) within the C-terminal active domain, as defined herein. In still another embodiment, the osteogenic proteins of the invention can be defined as osteogenically active proteins having any one of the generic sequences defined herein, including OPX (SEQ ID NO: 4) and Generic Sequences 7 (SEQ ID NO: 5) and 8 (SEQ ID NO: 6), or Generic Sequences 9 (SEQ ID NO: 7) and 10 (SEQ ID NO: 8).

The family of bone morphogenic polypeptides useful in the present invention, and members thereof, can be defined by a generic amino acid sequence. For example, Generic Sequence 7 (SEQ ID NO: 5) and Generic Sequence 8 (SEQ ID NO: 6) are 96 and 102 amino acid sequences, respectively, and accommodate the homologies shared among preferred protein family members identified to date, including at least OP-1, OP-2, OP-3, CBMP-2A, CBMP-2B, BMP-3, 60A, DPP, Vg1, BMP-5, BMP-6, Vgr-1, and GDF-1. The amino acid sequences for these proteins are described herein and/or in the art, as summarized above. The generic sequences include both the amino acid identity shared by these sequences in the C-terminal domain, defined by the six and seven cysteine skeletons (Generic Sequences 7 and 8, respectively), as well as alternative residues for the variable positions within the sequence. The generic sequences provide an appropriate cysteine skeleton where inter- or intramolecular disulfide bonds can form, and contain certain critical amino acids likely to influence the tertiary structure of the folded proteins. In addition, the generic sequences allow for an additional cysteine at position 36 (Generic Sequence 7) or position 41 (Generic Sequence 8), thereby encompassing the morphogenically active sequences of OP-2 and OP-3.

Generic Sequence 7

```
Leu Xaa Xaa Xaa Phe Xaa Xaa
                        1 5
Xaa Gly Trp Xaa Xaa Xaa Xaa Xaa Xaa Pro
                10 15
Xaa Xaa Xaa Xaa Ala Xaa Tyr Cys Xaa Gly
                20 25
Xaa Gys Xaa Xaa Pro Xaa Xaa Xaa Xaa Xaa
                30 35
Xaa Xaa Xaa Asn His Ala Xaa Xaa Xaa Xaa
                40 45
Xaa Xaa Xaa Xaa Xaa Xaa Xaa Xaa Xaa Xaa
                50 55
Xaa Xaa Xaa Cys Cys Xaa Pro Xaa Xaa Xaa
                60 65
Xaa Xaa Xaa Xaa Xaa Leu Xaa Xaa Xaa Xaa
                70 75
Xaa Xaa Xaa Val Xaa Leu Xaa Xaa Xaa Xaa
                80 85
Xaa Met Xaa Val Xaa Xaa Cys Xaa Cys Xaa
                90 95
```

wherein each Xaa independently is selected from a group of one or more specified amino acids defined as follows: “res.” means “residue” and Xaa at res.2=(Tyr or Lys); Xaa at res.3=Val or Ile); Xaa at res.4=(Ser, Asp or Glu); Xaa at res.6=(Arg, Gln, Ser, Lys or Ala); Xaa at res.7=(Asp or Glu); Xaa at res.8=(Leu, Val or Ile); Xaa at res. 11=(Gln, Leu, Asp, His, Asn or Ser); Xaa at res.12 =(Asp, Arg, Asn or Glu); Xaa at res.13=(Trp or Ser); Xaa at res.14=(Ile or Val); Xaa at res.15=(Ile or Val); Xaa at res.16(Ala or Ser); Xaa at res.18=(Glu, Gln, Leu, Lys, pro or Arg); Xaa at res.19=(Gly or Ser); Xaa at res.20=(Tyr or Phe); Xaa at res.21=(Ala, Ser, Asp, Met, His, Gln, Leu or Gly); Xaa at res.23=(Tyr, Asn or Phe); Xaa at res.26=(Glu, His, Tyr, Asp, Gln, Ala or Ser); Xaa at res.28=(Glu, Lys, Asp, Gln or Ala); Xaa at res.30=(Ala, Ser, Pro, Gln, Ile or Asn); Xaa at res.31=(Phe, Leu or Tyr); Xaa at res.33=(Leu, Val or Met); Xaa at res.34=(Asn, Asp, Ala, Thr or Pro); Xaa at res.35=(Ser, Asp, Glu, Leu, Ala or Lys); Xaa at res.36=(Tyr, Cys, His, Ser or Ile); Xaa at res.37=(Met, Phe, Gly or Leu); Xaa at res.38=(Asn, Ser or Lys); Xaa at res.39=(Ala, Ser, Gly or Pro); Xaa at res.40 =(Thr, Leu or Ser); Xaa at res.44=(Ile, Val or Thr); Xaa at res.45=(Val, Leu, Met or Ile); Xaa at res.46=(Gln or Arg); Xaa at res.47=(Thr, Ala or Ser); Xaa at res.48=(Leu or Ile); Xaa at res.49=(Val or Met); Xaa at res.50=(His, Asn or Arg); Xaa at res.51=(Phe, Leu, Asn, Ser, Ala or Val); Xaa at res.52=(Ile, Met, Asn, Ala, Val, Gly or Leu); Xaa at res.53=(Asn, Lys, Ala, Glu, Gly or Phe); Xaa at res.54=(Pro, Ser or Val); Xaa at res.55=(Glu, Asp, Asn, Gly, Val, Pro or Lys); Xaa at res.56=(Thr, Ala, Val, Lys, Asp, Tyr, Ser, Gly, Ile or His); Xaa at res.57=(Val, Ala or Ile); Xaa at res.58=(Pro or Asp); Xaa at res.59=(Lys, Leu or Glu); Xaa at res.60=(Pro, Val or Ala); Xaa at res.63=(Ala or Val); Xaa at res.65=(Thr, Ala or Glu); Xaa at res.66=(Gln, Lys, Arg or Glu); Xaa at res.67=(Leu, Met or Val); Xaa at res.68=(Asn, Ser, Asp or Gly); Xaa at res.69=(Ala, Pro or Ser); Xaa at res.70=(Ile, Thr, Val or Leu); Xaa at res.71=(Ser, Ala or Pro); Xaa at res.72=(Val, Leu, Met or Ile); Xaa at res.74=(Tyr or Phe); Xaa at res.75=(Phe, Tyr, Leu or His); Xaa at res.76=(Asp, Asn or Leu); Xaa at res.77=(Asp, Glu, Asn, Arg or Ser); Xaa at res.78=(Ser, Gln, Asn, Tyr or Asp); Xaa at res.79=(Ser, Asn, Asp, Glu or Lys); Xaa at res.80=(Asn, Thr or Lys); Xaa at res.82=(Ile, Val or Asn); Xaa at res.84=(Lys or Arg); Xaa at res.85=(Lys, Asn, Gln, His, Arg or Val); Xaa at res.86=(Tyr, Glu or His); Xaa at res.87=(Arg, Gln, Glu or Pro); Xaa at res.88=(Asn, Glu, Trp or Asp); Xaa at res.90=(Val, Thr, Ala or Ile); Xaa at res.92=(Arg, Lys, Val, Asp, Gln or Glu); Xaa at res.93=(Ala, Gly, Glu or Ser); Xaa at res.95=(Gly or Ala) and Xaa at res.97=(His or Arg).

Generic Sequence 8 (SEQ ID NO: 6) includes all of Generic Sequence 7 and in addition includes the following sequence (SEQ ID NO: 9) at its N-terminus:

SEQ ID NO:9

| Cys | Xaa | Xaa | Xaa | Xaa |
|---|---|---|---|---|
| 1 | | | | 5 |

Accordingly, beginning with residue 7, each “Xaa” in Generic Sequence 8 is a specified amino acid defined as for Generic Sequence 7, with the distinction that each residue number described for Generic Sequence 7 is shifted by five in Generic Sequence 8. Thus, “Xaa at res.2=(Tyr or Lys)” in Generic Sequence 7 refers to Xaa at res. 7 in Generic Sequence 8. In Generic Sequence 8, Xaa at res.2=(Lys, Arg, Ala or Gln); Xaa at res.3=(Lys, Arg or Met); Xaa at res.4=(His, Arg or Gln); and Xaa at res. 5=(Glu, Ser, His, Gly, Arg, Pro, Thr, or Tyr).

In another embodiment, useful osteogenic proteins include those defined by Generic Sequences 9 and 10, defined as follows.

Specifically, Generic Sequences 9 and 10 are composite amino acid sequences of the following proteins: human OP-1, human OP-2, human OP-3, human BMP-2, human BMP-3, human BMP-4, human BMP-5, human BMP-6, human BMP-8, human BMP-9, human BMP 10, human BMP-11, Drosophila 60A, Xenopus Vg-1, sea urchin UNIVIN, human CDMP-1 (mouse GDF-5), human CDMP-2 (mouse GDF-6, human BMP-13), human CDMP-3 (mouse GDF-7, human BMP-12), mouse GDF-3, human GDF-1, mouse GDF-1, chicken DORSALIN, dpp, Drosophila SCREW, mouse NODAL, mouse GDF-8, human GDF-8, mouse GDF-9, mouse GDF-10, human GDF-11, mouse GDF-11, human BMP-15, and rat BMP3b. Like Generic Sequence 7, Generic Sequence 9 is a 96 amino acid sequence that accommodates the C-terminal six cysteine skeleton and, like Generic Sequence 8, Generic Sequence 10 is a 102 amino acid sequence which accommodates the seven cysteine skeleton.

```
Generic Sequence 9
Xaa Xaa Xaa Xaa Xaa Xaa Xaa Xaa Xaa Xaa
1 5 10
Xaa Xaa Xaa Xaa Xaa Xaa Pro Xaa Xaa Xaa
                                15 20
Xaa Xaa Xaa Xaa Cys Xaa Gly Xaa Cys Xaa
                                25 30
Xaa Xaa Xaa Xaa Xaa Xaa Xaa Xaa Xaa Xaa
                                35 40
Xaa Xaa Xaa Xaa Xaa Xaa Xaa Xaa Xaa Xaa
                                45 50
Xaa Xaa Xaa Xaa Xaa Xaa Xaa Xaa Xaa Xaa
                                55 60
Xaa Cys Xaa Pro Xaa Xaa Xaa Xaa Xaa Xaa
                                65 70
Xaa Xaa Leu Xaa Xaa Xaa Xaa Xaa Xaa Xaa
                                75 80
Xaa Xaa Xaa Xaa Xaa Xaa Xaa Xaa Xaa Xaa
                                85 90
Xaa Xaa Xaa Cys Xaa Cys Xaa
                                95
```

wherein each Xaa is independently selected from a group of one or more specified amino acids defined as follows: “res.” means “residue” and Xaa at res. 1=(Phe, Leu or Glu); Xaa at res. 2=(Tyr, Phe, His, Arg, Thr, Lys, Gin, Val or Glu); Xaa at res. 3=(Val, Ile, Leu or Asp); Xaa at res. 4=(Ser, Asp, Glu, Asn or Phe); Xaa at res. 5=(Phe or Glu); Xaa at res. 6=(Arg, Gln, Lys, Ser, Glu, Ala or Asn); Xaa at res. 7=(Asp, Glu, Leu, Ala or Gln); Xaa at res. 8=(Leu, Val, Met, Ile or Phe); Xaa at res. 9=(Gly, His or Lys); Xaa at res. 10=(Trp or Met); Xaa at res. 11=(Gln, Leu, His, Glu, Asn, Asp, Ser or Gly); Xaa at res. 12=(Asp, Asn, Ser, Lys, Arg, Glu or His); Xaa at res. 13=(Trp or Ser); Xaa at res. 14=(Ile or Val); Xaa at res. 15=(Ile or Val); Xaa at res. 16=(Ala, Ser, Tyr or Trp); Xaa at res. 18=(Glu, Lys, Gln, Met, Pro, Leu, Arg, His or Lys); Xaa at res. 19=(Gly, Glu, Asp, Lys, Ser, Gln, Arg or Phe); Xaa at res. 20=(Tyr or Phe); Xaa at res. 21=(Ala, Ser, Gly, Met, Gln, His, Glu, Asp, Leu, Asn, Lys or Thr); Xaa at res. 22=(Ala or Pro); Xaa at res. 23=(Tyr, Phe, Asn, Ala or Arg); Xaa at res. 24=(Tyr, His, Glu, Phe or Arg); Xaa at res. 26=(Glu, Asp, Ala, Ser, Tyr, His, Lys, Arg, Gln or Gly); Xaa at res. 28=(Glu, Asp, Leu, Val, Lys, Gly, Thr, Ala or Gln); Xaa at res. 30=(Ala, Ser, Ile, Asn, Pro, Glu, Asp, Phe, Gln or Leu); Xaa at res. 31=(Phe, Tyr, Leu, Asn, Gly or Arg); Xaa at res. 32=(Pro, Ser, Ala or Val); Xaa at res. 33=(Leu, Met, Glu, Phe or Val); Xaa at res. 34=(Asn, Asp, Thr, Gly, Ala, Arg, Leu or Pro); Xaa at res. 35=(Ser, Ala, Glu, Asp, Thr, Leu, Lys, Gln or His); Xaa at res. 36=(Tyr, His, Cys, Ile, Arg, Asp, Asn, Lys, Ser, Glu or Gly); Xaa at res. 37=(Met, Leu, Phe, Val, Gly or Tyr); Xaa at res. 38=(Asn, Glu, Thr, Pro, Lys, His, Gly, Met, Val or Arg); Xaa at res. 39=(Ala, Ser, Gly, Pro or Phe); Xaa at res. 40=(Thr, Ser, Leu, Pro, His or Met); Xaa at res. 41=(Asn, Lys, Val, Thr or Gln); Xaa at res. 42 =(His, Tyr or Lys); Xaa at res. 43=(Ala, Thr, Leu or Tyr); Xaa at res. 44=(Ile, Thr, Val, Phe, Tyr, Met or Pro); Xaa at res. 45=(Val, Leu, Met, Ile or His); Xaa at res. 46=(Gln, Arg or Thr); Xaa at res. 47=(Thr, Ser, Ala, Asn or His); Xaa at res. 48=(Leu, Asn or Ile); Xaa at res. 49=(Val, Met, Leu, Pro or Ile); Xaa at res. 50=(His, Asn, Arg, Lys, Tyr or Gln); Xaa at res. 51=(Phe, Leu, Ser, Asn, Met, Ala, Arg, Glu, Gly or Gln); Xaa at res. 52=(Ile, Met, Leu, Val, Lys, Gln, Ala or Tyr); Xaa at res. 53=(Asn, Phe, Lys, Glu, Asp, Ala, Gln, Gly, Leu or Val); Xaa at res. 54=(Pro, Asn, Ser, Val or Asp); Xaa at res. 55=(Glu, Asp, Asn, Lys, Arg, Ser, Gly, Thr, Gln, Pro or His); Xaa at res. 56=(Thr, His, Tyr, Ala, Ile, Lys, Asp, Ser, Gly or Arg); Xaa at res. 57=(Val, Ile, Thr, Ala, Leu or Ser); Xaa at res. 58=(Pro, Gly, Ser, Asp or Ala); Xaa at res. 59=(Lys, Leu, Pro, Ala, Ser, Glu, Arg or Gly); Xaa at res. 60=(Pro, Ala, Val, Thr or Ser); Xaa at res. 61=(Cys, Val or Ser); Xaa at res. 63=(Ala, Val or Thr); Xaa at res. 65=(Thr, Ala, Glu, Val, Gly, Asp or Tyr); Xaa at res. 66=(Gln, Lys, Glu, Arg or Val); Xaa at res. 67=(Leu, Met, Thr or Tyr); Xaa at res. 68=(Asn, Ser, Gly, Thr, Asp, Glu, Lys or Val); Xaa at res. 69 =(Ala, Pro, Gly or Ser); Xaa at res. 70=(Ile, Thr, Leu or Val); Xaa at res. 71=(Ser, Pro, Ala, Thr, Asn or Gly); Xaa at res. 2=(Val, Ile, Leu or Met); Xaa at res. 74=(Tyr, Phe, Arg, Thr, Tyr or Met); Xaa at res. 75=(Phe, Tyr, His, Leu, Ile, Lys, Gln or Val); Xaa at res. 76=(Asp, Leu, Asn or Glu); Xaa at res. 77=(Asp, Ser, Arg, Asn, Glu, Ala, Lys, Gly or Pro); Xaa at res. 78=(Ser, Asn, Asp, Tyr, Ala, Gly, Gln, Met, Glu, Asn or Lys); Xaa at res. 79=(Ser, Asn, Glu, Asp, Val, Lys, Gly, Gln or Arg); Xaa at res. 80=(Asn, Lys, Thr, Pro, Val, Ile, Arg, Ser or Gln); Xaa at res. 81=(Val, Ile, Thr or Ala); Xaa at res. 82=(Ile, Asn, Val, Leu, Tyr, Asp or Ala); Xaa at res. 83=(Leu, Tyr, Lys or Ile); Xaa at res. 84=(Lys, Arg, Asn, Tyr, Phe, Thr, Glu or Gly); Xaa at res. 85=(Lys, Arg, His, Gln, Asn, Glu or Val); Xaa at res. 86=(Tyr, His, Glu or Ile); Xaa at res. 87=(Arg, Glu, Gln, Pro or Lys); Xaa at res. 88=(Asn, Asp, Ala, Glu, Gly or Lys); Xaa at res. 89=(Met or Ala); Xaa at res. 90=(Val, Ile, Ala, Thr, Ser or Lys); Xaa at res 91=(Val or Ala); Xaa at res. 92=(Arg, Lys, Gln, Asp, Glu, Val, Ala, Ser or Thr); Xaa at res. 93=(Ala, Ser, Glu, Gly, Arg or Thr); Xaa at res. 95=(Gly, Ala or Thr); Xaa at res. 97=(His, Arg, Gly, Leu or Ser). Further, after res. 53 in rBMP3b and mGDF-10 there is an Ile; after res. 54 in GDF-1 there is a T ; after res. 54 in BMP3 there is a V; after res. 78 in BMP-8 and Dorsalin there is a G; after res. 37 in hGDF-1 there is Pro, Gly, Gly, Pro.

Generic Sequence 10 (SEQ ID NO: 8) includes all of Generic Sequence 9 (SEQ ID NO: 7) and in addition includes the following sequence (SEQ ID NO: 9) at its N-terminus:

SEQ ID NO:9

| Cys | Xaa | Xaa | Xaa | Xaa |
|---|---|---|---|---|
| 1 | | | | 5 |

Accordingly, beginning with residue 6, each “Xaa” in Generic Sequence 10 is a specified amino acid defined as for Generic Sequence 9, with the distinction that each residue number described for Generic Sequence 9 is shifted by five in Generic Sequence 10. Thus, “Xaa at res. 1=( Tyr, Phe, His, Arg, Thr, Lys, Gln, Val or Glu)” in Generic Sequence 9 refers to Xaa at res. 6 in Generic Sequence 10. In Generic Sequence 10, Xaa at res. 2=(Lys, Arg, Gln, Ser, His, Glu, Ala, or Cys); Xaa at res. 3=(Lys, Arg, Met, Lys, Thr, Leu, Tyr, or Ala); Xaa at res. 4=(His, Gin, Arg, Lys, Thr, Leu, Val, Pro, or Tyr); and Xaa at res. 5=(Gln, Thr, His, Arg, Pro, Ser, Ala, Gln, Asn, Tyr, Lys, Asp, or Leu).

As noted above, certain currently preferred bone morphogenic polypeptide sequences useful in this invention have greater than 60% identity, preferably greater than 65%, 70%, 75%, 80%, 85%, 90%, 95%, or 98% identity, with the amino acid sequence defining the preferred reference sequence of hOP-1. These particularly preferred sequences include allelic and phylogenetic counterpart variants of the OP-1 and OP-2 proteins, including the Drosophila 60A protein. Accordingly, in certain particularly preferred embodiments, useful morphogenic proteins include active proteins comprising pairs of polypeptide chains within the generic amino acid sequence herein referred to as “OPX” (SEQ ID NO: 4), which defines the seven cysteine skeleton and accommodates the homologies between several identified variants of OP-1 and OP-2. As described therein, each Xaa at a given position independently is selected from the residues occurring at the corresponding position in the C-terminal sequence of mouse or human OP-1 or OP-2.

```
Cys Xaa Xaa His Glu Leu Tyr Val Ser Phe Xaa Asp Leu Gly Trp Xaa Asp Trp
1 5 10 15
Xaa Ile Ala Pro Xaa Gly Tyr Xaa Ala Tyr Tyr Cys Glu Gly Glu Cys Xaa Phe Pro
  20 25 30 35
Leu Xaa Ser Xaa Met Asn Ala Thr Asn His Ala Ile Xaa Gln Xaa Leu Val His Xaa
          40 45 50 55
Xaa Xaa Pro Xaa Xaa Val Pro Lys Xaa Cys Cys Ala Pro Thr Xaa Leu Xaa Ala
                          60 65 70
Xaa Ser Val Leu Tyr Xaa Asp Xaa Ser Xaa Asn Val Ile Leu Xaa Lys Xaa Arg
75 80 85 90
Asn Met Val Val Xaa Ala Cys Gly Cys His
        95 100
```

wherein Xaa at res. 2=(Lys or Arg); Xaa at res. 3=(Lys or Arg); Xaa at res. 11=(Arg or Gln); Xaa at res. 16=(Gln or Leu); Xaa at res. 19=(Ile or Val); Xaa at res. 23=(Glu or Gln); Xaa at res. 26=(Ala or Ser); Xaa at res. 35=(Ala or Ser); Xaa at res. 39=(Asn or Asp); Xaa at res. 41=(Tyr or Cys); Xaa at res. 50=(Val or Leu); Xaa at res. 52=(Ser or Thr); Xaa at res. 56=(Phe or Leu); Xaa at res. 57 =(Ile or Met); Xaa at res. 58=(Asn or Lys); Xaa at res. 60=(Glu, Asp or Asn); Xaa at res. 61=(Thr, Ala or Val); Xaa at res. 65=(Pro or Ala); Xaa at res. 71=(Gln or Lys); Xaa at res. 73=(Asn or Ser); Xaa at res. 75=(Ile or Thr); Xaa at res. 80=(Phe or Tyr); Xaa at res. 82=(Asp or Ser); Xaa at res. 84=(Ser or Asn); Xaa at res. 89=(Lys or Arg); Xaa at res. 91=(Tyr or His); and Xaa at res. 97=(Arg or Lys).

In still another preferred embodiment, useful osteogenically active proteins have polypeptide chains with amino acid sequences comprising a sequence encoded by a nucleic acid that hybridizes, under low, medium or high stringency hybridization conditions, to DNA or RNA encoding reference morphogen sequences, e.g., C-terminal sequences defining the conserved seven cysteine domains of OP-1, OP-2, BMP-2, BMP-4, BMP-5, BMP-6, 60A, GDF-3, GDF-6, GDF-7 and the like. As used herein, high stringent hybridization conditions are defined as hybridization according to known techniques in 40% formamide, 5×SSPE, 5× Denhardt's Solution, and 0.1% SDS at 37° C. overnight, and washing in 0.1×SSPE, 0.1% SDS at 50° C. Standard stringent conditions are well characterized in commercially available, standard molecular cloning texts. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984): Nucleic Acid Hybridization (B. D. Hames & S. J. Higgins eds. 1984); and B. Perbal, A Practical Guide To Molecular Cloning (1984), the disclosures of which are incorporated herein by reference.

As noted above, proteins useful in the present invention generally are dimeric proteins comprising a folded pair of the above polypeptides. Such morphogenic proteins are inactive when reduced, but are active as oxidized homodimers and when oxidized in combination with others of this invention to produce heterodimers. Thus, members of a folded pair of morphogenic polypeptides in a morphogenically active protein can be selected independently from any of the specific polypeptides mentioned above. In some embodiments, the bone morphogenic protein is a monomer.

The bone morphogenic proteins useful in the materials and methods of this invention include proteins comprising any of the polypeptide chains described above, whether isolated from naturally-occurring sources, or produced by recombinant DNA or other synthetic techniques, and includes allelic and phylogenetic counterpart variants of these proteins, as well as muteins thereof, and various truncated and fusion constructs. Deletion or addition mutants also are envisioned to be active, including those which may alter the conserved C-terminal six or seven cysteine domain, provided that the alteration does not functionally disrupt the relationship of these cysteines in the folded structure. Accordingly, such active forms are considered the equivalent of the specifically described constructs disclosed herein. The proteins may include forms having varying glycosylation patterns, varying N-termini, a family of related proteins having regions of amino acid sequence homology, and active truncated or mutated forms of native or biosynthetic proteins, produced by expression of recombinant DNA in host cells.

The bone morphogenic proteins contemplated herein can be expressed from intact or truncated cDNA or from synthetic DNAs in prokaryotic or eukaryotic host cells, and purified, cleaved, refolded, and dimerized to form morphogenically active compositions. Currently preferred host cells include, without limitation, prokaryotes including E. coli or eukaryotes including yeast, or mammalian cells, such as CHO, COS or BSC cells. One of ordinary skill in the art will appreciate that other host cells can be used to advantage. Detailed descriptions of the bone morphogenic proteins useful in the practice of this invention, including how to make, use and test them for osteogenic activity, are disclosed in numerous publications, including U.S. Pat. Nos. 5,266,683 and 5,011,691, the disclosures of which are incorporated by reference herein, as well as in any of the publications recited herein, the disclosures of which are incorporated herein by reference.

Thus, in view of this disclosure and the knowledge available in the art, skilled genetic engineers can isolate genes from cDNA or genomic libraries of various different biological species, which encode appropriate amino acid sequences, or construct DNAs from oligonucleotides, and then can express them in various types of host cells, including both prokaryotes and eukaryotes, to produce large quantities of active proteins capable of stimulating bone and cartilage morphogenesis in a mammal.

In some embodiments, the osteogenic protein includes, but is not limited to OP-1, OP-2, OP-3, BMP-2, BMP-3, BMP-4, BMP-5, BMP-6, ,BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and amino acid sequence variants thereof. In some embodiments, the osteogenic protein comprises an amino acid sequence having at least 70% homology with the C-terminal 102-106 amino acids, including the conserved seven cysteine domain, of human OP-1, said osteogenic protein being capable of inducing repair of the cartilage defect.

In a preferred embodiment, the morphogenic protein is OP-1, GDF-5, GDF-6 and GDF-7, CDMP-1, CDMP-2 or CDMP-3. In a most preferred embodiment, the morphogenic protein is OP-1.

Pharmaceutical Compositions

The pharmaceutical compositions comprising a morphogenic protein may be in a variety of forms. These include, for example, solid, semisolid and liquid dosage forms such as powders, tablets, pills, suppositories, liquid solutions, suspensions, gels, putty, pastes, emulsions and infusible solutions. The preferred form depends on the intended mode of administration and the therapeutic application and may be selected by one skilled in the art. Modes of administration may include oral, parenteral, intramuscular, intraperitoneal, intra-articular, subcutaneous, intravenous, intralesional or topical administration. The compositions may be formulated in dosage forms appropriate for each route of administration. In some embodiments, the pharmaceutical compositions of this invention will be administered into the site (i.e., directly into the cartilage) in need of tissue regeneration or repair. In other embodiments, the pharmaceutical compositions of this invention will be administered in the vicinity of the site in need of tissue regeneration or repair. For example, in some embodiments, the pharmaceutical compositions of this invention may be administered into the area surrounding the cartilage (e.g., the synovial fluid) in need of repair (i.e. a joint). In other embodiments, the pharmaceutical compositions of this invention may be administered directly into the cartilage tissue (e.g., a meniscus or an intervertebral disc).

The pharmaceutical compositions comprising a morphogenic protein may, for example, be placed into sterile, isotonic formulations with or without cofactors which stimulate uptake or stability. The formulation is preferably liquid, or may be lyophilized powder. For example, the morphogenic protein may be diluted with a formulation buffer. The solution can be lyophilized, stored under refrigeration and reconstituted prior to administration with sterile Water-For-Injection (USP).

The compositions also will preferably include conventional pharmaceutically acceptable carriers well known in the art (see, e.g., Remington's Pharmaceutical Sciences, 16th Ed., Mac Publishing Company (1980)). Such pharmaceutically acceptable carriers may include other medicinal agents, carriers, genetic carriers, adjuvants, excipients, etc., such as human serum albumin or plasma preparations. Preferably, the carrier is isotonic with the blood or synovial fluid of the patient. Examples of such carrier vehicles include water, saline, Ringer's solution, a buffered solution, hyaluronan and dextrose solution. Non-aqueous vehicles such as fixed oils and ethyl oleate are also useful herein. The compositions are preferably in the form of a unit dose and will usually be administered as a dose regimen that depends on the particular tissue treatment.

In some embodiments, the compositions of this invention are sustained release formulations, slow delivery formulations, formulations whereby the morphogenic protein clearance is delayed. There are numerous delivery materials available for preparing these compositions. They include, but are not limited to, microspheres of polylactic/polyglycolic acid polymers, liposomes, collagen, polyethylene glycol (PEG), hyaluronic acid/fibrin matrices, hyaluronic acid, fibrin, chitosan, gelatin, SABER™ System (sucrose acetate isobutyrate (SAIB)), DURIN™ (biodegradabale polymer for drug loaded implants), MICRODUR™ (biodegradable polymers/microencapsulation) and DUROS™ (mini-osmotic pump). In some embodiments, the morphogenic protein is covalently linked to the delivery material.

In a preferred embodiment, the morphogenic protein is formulated with polyethylene glycol (PEG) as a delivery material. The PEG group(s) may be of any convenient molecular weight and may be linear or branched. In some embodiments, the morphogenic composition comprises PEG. In some embodiments, the morphogenic protein is covalently linked to the PEG group(s). Generally, PEG group(s) are connected to the morphogenic protein via a reactive group on the morphogenic protein and the PEG group. Methods for PEGylating proteins are known in the art. In a preferred embodiment, the morphogenic protein used in the PEG containing formulation includes but is not limited to OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and amino acid sequence variants thereof. In a more preferred embodiment, the formulation is PEGylated OP-1.

The compositions of this invention comprise a morphogenic protein dispersed in a biocompatible carrier material that functions as a suitable delivery system for the compounds. Suitable examples of sustained release carriers include semipermeable polymer matrices. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers, 22, pp. 547-56 (1985)); poly(2-hydroxyethyl-methacrylate), ethylene vinyl acetate (Langer et al., J. Biomed. Mater. Res., 15, pp. 167-277 (1981); Langer, Chem. Tech., 12, pp. 98-105 (1982)) or poly-D-(−)-3hydroxybutyric acid (EP 133,988), polylactic acid, poly glycolic acid or polymers of the above.

The pharmaceutical compositions of this invention may also be administered using, for example, microspheres, liposomes, other microparticulate delivery systems or sustained release formulations placed in, near, or otherwise in communication with affected tissues, the fluids bathing those tissues (e.g., synovial fluid) or bloodstream bathing those tissues.

Liposomes containing a morphogenic protein of this invention can be prepared by well-known methods (See, e.g. D E 3,218,121; Epstein et al., Proc. Natl. Acad. Sci. U.S.A., 82, pp. 3688-92 (1985); Hwang et al., Proc. Natl. Acad. Sci. U.S.A., 77, pp. 4030-34 (1980); U.S. Pat. Nos. 4,485,045 and 4,544,545). Ordinarily the liposomes are of the small (about 200-800 Angstroms) unilamellar type in which the lipid content is greater than about 30 mol. % cholesterol. The proportion of cholesterol is selected to control the optimal rate of morphogenic protein release.

The morphogenic proteins of this invention may also be attached to liposomes containing other biologically active molecules such as immunosuppressive agents, cytokines, etc., to modulate the rate and characteristics of tissue induction. Attachment of morphogenic proteins to liposomes may be accomplished by any known cross-linking agent such as heterobifunctional cross-linking agents that have been widely used to couple toxins or chemotherapeutic agents to antibodies for targeted delivery. Conjugation to liposomes can also be accomplished using the carbohydrate-directed cross-linking reagent 4-(4-maleimidophenyl) butyric acid hydrazide (MPBH) (Duzgunes et al., J. Cell. Biochem. Abst. Suppl. 16E 77 (1992)).

The morphogenic proteins of this invention may also be glycosylated. Glycosylation is the modification of a protein by addition of one or more oligosaccharide groups. There are usually two types of glycosylation: O-linked oligosaccharides are attached to serine or threonine residues while N-linked oligosaccharides are attached to asparagine residues when they are part of the sequence Asn-X-Ser/Thr, where X can be any amino acid except proline. Glycosylation can dramatically affect the physical properties of proteins and can also be important in protein stability, secretion, half-life, and subcellular localization. In some embodiments, the morphogenic proteins of the present invention comprise N-linked oligosaccharaides. In other embodiments, the morphogenic proteins of this invention comprise O-linked oligosaccharides. In yet other embodiments, the morphogenic proteins of this inventions comprise both N-linked and O-linked oligosaccharides. In some embodiments, the glycosylation pattern of the morphogenic protein may be modified to control the carbohydrate composition of the glycoprotein.

One skilled in the art may create a biocompatible, and or biodegradable formulation of choice to promote tissue induction.

A successful carrier for morphogenic proteins should perform several important functions. It should act as a slow release delivery system of morphogenic protein or delay clearance of the morphogenic protein, and protect the morphogenic protein from non-specific proteolysis.

In addition, selected materials must be biocompatible in vivo and preferably biodegradable. Polylactic acid (PLA), polyglycolic acid (PGA), and various combinations have different dissolution rates in vivo.

The carrier may also take the form of a hydrogel. When the carrier material comprises a hydrogel, it refers to a three dimensional network of cross-linked hydrophilic polymers in the form of a gel substantially composed of water, preferably but not limited to gels being greater than 90% water. Hydrogel can carry a net positive or net negative charge, or may be neutral. A typical net negative charged hydrogel is alginate. Hydrogels carrying a net positive charge may be typified by extracellular matrix components such as collagen and laminin. Examples of commercially available extracellular matrix components include Matrigel™ and Vitrogen™. An example of a net neutral hydrogel is highly crosslinked polyethylene oxide, or polyvinyalcohol.

Various growth factors, cytokines, hormones, trophic agents and therapeutic compositions including antibiotics and chemotherapeutic agents, enzymes, enzyme inhibitors and other bioactive agents also may be adsorbed onto or dispersed within the carrier material comprising the morphogenic protein, and will also be released over time and is slowly absorbed.

Dosage levels of between about 1 μg and about 1000 μg per day, preferably between 3 μg and 50 μg per day of the morphogenic protein are useful in cartilage repair and regeneration. As the skilled artisan will appreciate, lower or higher doses than those recited may be required. Specific dosage and treatment regimens for any particular patient will depend upon a variety of factors, including the activity of the specific morphogenic protein employed, the age, body weight, general health status, sex, diet, time of administration, rate of excretion, drug combination, the severity of the tissue damage and the judgment of the treating physician.

### EXAMPLE 1

### Dog Model Repair of Osteochondral Defects

12 adult male bred for purpose dogs will undergo surgery. Both hindlimbs will be prepped and draped in sterile fashion. A medial parapatellar incision approximately four centimeters in length will be made. The patella will be retracted laterally to expose the femoral condyle. In the right medial condyle, a 5.0 mm diameter defect extending through the cartilage layer and penetrating the subchondral bone to a depth of 6 mm will be created in the central load bearing region of the femoral condyle with a specially designed or modified 5.0 mm drill bit. The animals will be divided into two groups of 6 animals each. In the first group, after copious irrigation with saline to remove debris and spilled marrow cells, the appropriate time release OP-1 will be applied to the synovial fluid surrounding the defect. In the first group of 6 animals, the right defects will receive the time release OP-1. The left limb of all animals will serve as a control receiving control beads (0% OP-1).

The second group of 6 animals will receive no OP-1 treatment at the time of surgery. At 3 days post surgery, the appropriate time release OP-1 formulation will be injected into the synovial fluid surrounding the joint with the defect. In 6 animals, time release OP-1 will be injected into the synovial fluid around the right defect. The left limb of all animals will serve as a control receiving control beads (0% OP-1).

The animals will be sacrificed at 16 weeks post-surgery. At sacrifice, the distal femurs will be retrieved en bloc and the defect sites will be evaluated histologically and grossly based on the scheme of Moran et al (J. Bone Joint Surg. 74B:659-667, 1992) that has been used in previous investigations.

Radiographs of the hindlimbs will be obtained preoperatively, immediately postoperative, and at postoperative week 16. The preoperative radiographs will be used to assure that no pre-existing abnormalities are present and to verify skeletal maturity. Post-operative radiographs will be used to assess defect placement. Sacrifice radiographs will be used to assess the rate of healing and restoration of the subchondral bone and the articulating surface. Radiographs will be obtained within one week of the evaluation date.

Gross pathological examination of the carcasses will be conducted immediately after sacrifice. The distal femurs will be immediately harvested en bloc and stored in saline soaked towels and placed in labeled plastic bags. High power photographs of the defect sites will be taken and carefully labeled.

Soft tissues will be meticulously dissected away from the defect site and the proximal end of the femur will be removed. On a water cooled diamond cut saw each defect site will be isolated for histologic evaluation.

Specimens will be fixed by immersion in 4% paraformaldehyde solution and prepared for decalcified histologic processing. Three sections from three levels will be cut from each block. Levels 1 and 3 will be closest to the defect perimeter. Level 2 will be located at the defect center. Three sections from each level will be stained with toluidine blue and Safranin O and fast green. Sections will be graded based on the scheme of Moran et al. (J. Bone Joint Surg. 74B:659-667, 1992).

It is expected that OP-1 treated animals will exhibit improved repair of the osteochondral defects when compared to control animals.

### EXAMPLE 2

### Sheep Model of Regeneration of Chondral Defects By Intra-Articular Administration of OP-1 in Time-Release Microspheres

18 adult bred for purpose sheep will undergo surgery. With a specially designed instrument, a 10 mm chondral defect will be created in the left hindlimb knee of 18 sheep on the weight bearing condyle surface, 2 mm deep up to the calcified layer (exposition of blood will be pronounced as a failure). The right knees of all animals will remain untouched to serve as a control.

Group 1 (6 animals): At postoperative day 3, the left knee of each animal will receive an intra-articular injection of a 250 μl suspension containing 57 mg of control 0.3% microspheres without OP-1.

Group 2 (6 animals): At postoperative day 3, the left knee of each animal will receive an intra-articular injection of a 250 μl suspension containing 57 mg of 0.3% microspheres containing 170 kg of OP-1.

Group 3 (6 animals): At postoperative day 3 and at postoperative week 6, the left knee of each animal will receive an intra-articular injection of a 250 μl suspension containing 57 mg of 0.3% microspheres containing 170 kg of OP-1.

Arthroscopic evaluation of the knees will be performed at postoperative weeks 3 and 6 on all the animals. NMR/MRI scans will be performed at postoperative week 3 and 6. Mechanical testing of the knees will also be performed periodically.

All animals will be sacrificed at 3 months postoperative. After sacrifice, histology, histomorphometry, immunostaining, and in situ hybridization for specific articular chondrocyte markers will be performed. It is expected that OP-1 treated knees will exhibit improved regeneration when compared to control knees.

### EXAMPLE 3

### Sheep Model for Prevention of Osteoarthritis

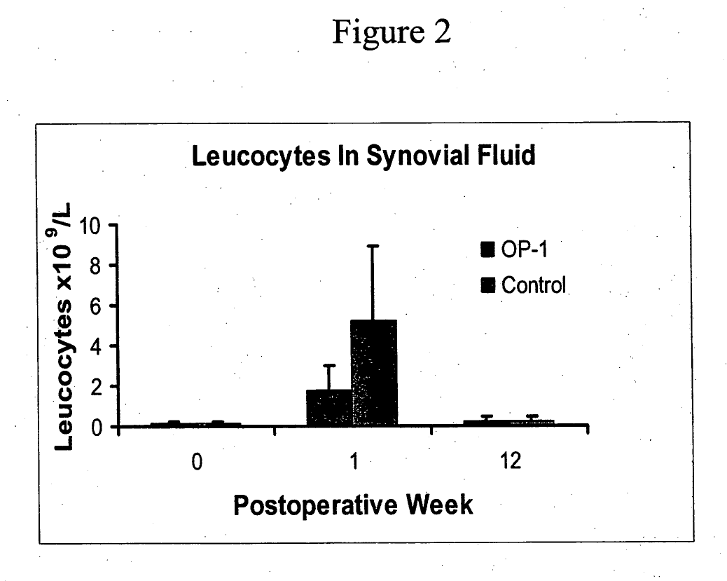

Sheep are used as a model for osteoarthritis because it has been demonstrated that progressive osteoarthritis occurs in these animals after a single injury impact. Twelve adult female crossbred sheep that are acclimatized for 14 days were used in this study. All sheep received general anesthesia and using aseptic techniques, a 3 cm arthrotomy was used to allow access to both femorotibial joints. A spring loaded mechanical device was used to create bilateral impact injuries to the weight bearing region of the median femoral condyle (30 Mpa, 6 mm diameter×2) (see FIG. 1). After a routine closure of these incisions, the sheep received an intra-articular injection in each knee of OP-1 in a vehicle of collagen and carboxymethylcellulose (OP-1 Implant, 340 μg) or vehicle alone. Two experimental groups (N=6) were used. Group A received 0.3 ml of OP-1+collagen+carboxymethylcellulose intra-articularly in one knee at the time of surgery (day 0) and one week later (day 7). Day 0 injections were administered immediately after the surgical incision is closed. Group B received OP-1 in one knee on day 0, 7, 14, 21, 28, and 35. Synovial fluid was aspirated before injection of the OP-1 and vehicle to allow measurement of leukocyte numbers and total protein as indicators of inflammation. OP-1 treatment significantly reduced leucocytes in synovial fluid 1 week postoperatively (p<.003, paired T test) but not total protein concentration (see FIG. 2).

The sheep were sacrificed 12 weeks postoperatively for detailed assessment (paravital staining, TUNEL staining, histopathology, cartilage, sulfated GAG analysis, biomechanical indentation testing) of the articular tissues. Abnormal cartilage (India ink uptake) was significantly different between groups ((p<.03) because lesions in OP-1 knees were often limited to reduced sheen/reflectivity whereas control joints had areas of fibrillation or erosion (see table 1).

TABLE 1

Abnormal Cartilage %¹

| Animal | Vehicle | OP-1 |
|---|---|---|
| 28 | 20 | 5 |
| 29 | 40 | 20 |
| 30 | 60 | 0 |
| 31 | 50 | 20 |
| 32 | 70 | 10 |
| 33 | 25 | 10 |

¹From India ink uptake on joint surface, digital photography, scaled area measurements using Northern Eclipse ™ morphometry software.

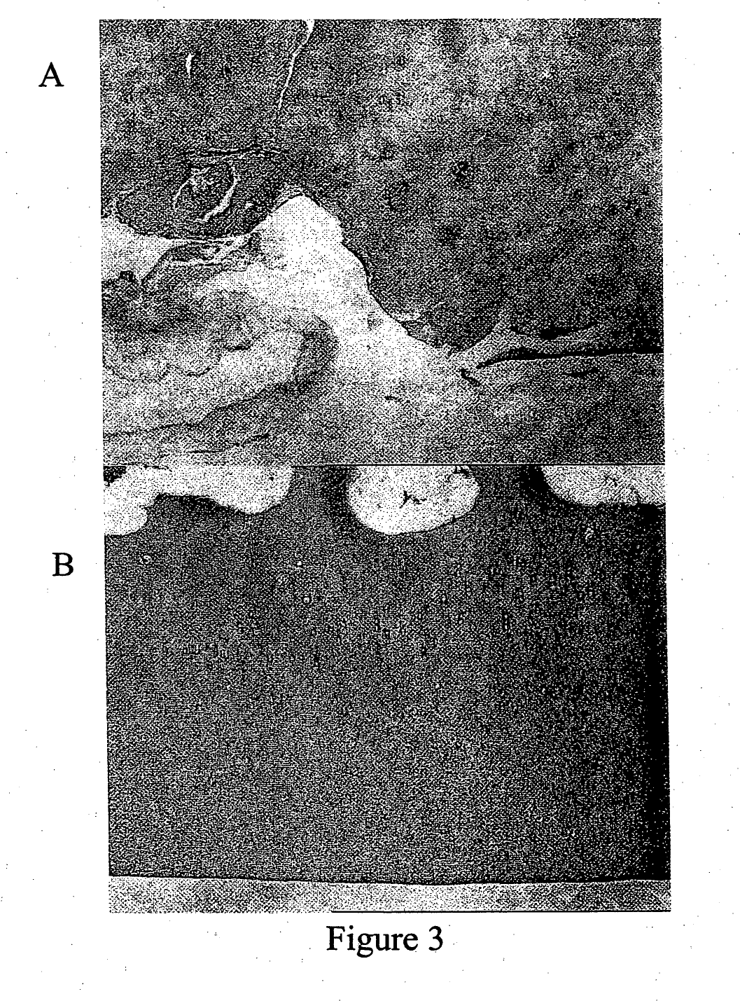

Histological sections showed chondrocyte clusters, acellular matrix and cartilage loss in vehicle treated joints (FIG. 3A), whereas lesions in OP-1 treated joints (FIG. 3B) were superficial zone chondrocyte loss and/or small fissures. Mankin histology scoring was not significantly different (p<.06, Wilcoxon Signed Rank Test), but the OARSI scoring system that is sensitive to the size of the lesion proved valuable (p<.03) (see table 2) (van der Sluijs J. et al., The reliability of the Mankin score for osteoarthrits. Ortho Res 1992, 10:58-61).

TABLE 2

| | |
|---|---|
| Modified Mankin Score¹ | OARSI Score² |

| Animal | Vehicle | OP-1 | Vehicle | OP-1 |
|---|---|---|---|---|
| 28 | 4 | 2 | 6 | 2 |
| 29 | 4 | 2 | 8 | 1.5 |
| 30 | 4 | 2 | 8 | 1 |
| 31 | 5 | 1 | 12 | 5 |
| 32 | 4 | 3 | 13.5 | 4 |
| 33 | 6 | 3 | 10 | 4 |

¹Modified Mankin score is 0-13 where 0 is normal cartilage.
²Osteoarthritis Research Society International Score calculation = lesion severity × area with a maximum of 24 for a single lesion.

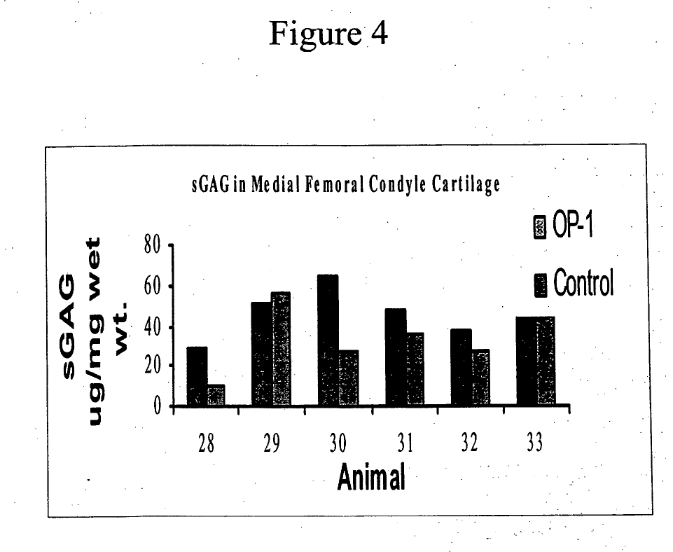

Sulfated glycosaminoglycan concentrations were higher in the OP-1 treated group with a strong trend toward statistical significance (p<.06) (see FIG. 4).

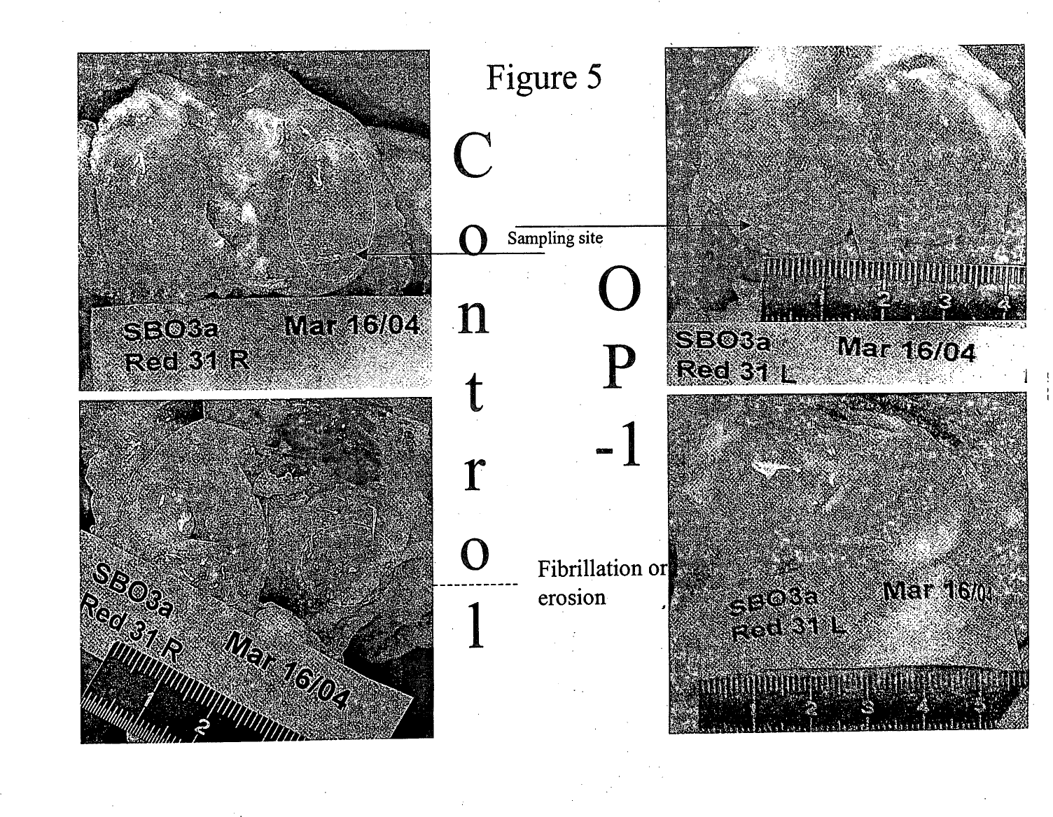

The collagen/CMC alone group resulted in fibrillations and erosion of the surface, whereas the OP-1 group shows little or no sign of damage (see FIG. 5). The OP-1 treated joints look healthier and shinier than the controls.

These experiments demonstrate marked improvement, if not complete protection with two injections of OP-1. Small lesions may persist in the face of therapy because 30 MPa impact injuries partial thickness defects may occur that are unlikely to repair completely. OP-1 was able to suppress the centrifugal extension of degenerative changes over the femoral condyle, whereas vehicle treated joints developed a unicompartmental osteoarthritis. The mechanism by which OP-1 exerts this effect may be through its anabolic properties by affecting repair. However, little repair tissue was present at the impacted sites so another mechanism that promotes survival of injured chondrocytes may be operative. These observations indicate that OP-1 may be useful for other applications such as tissue engineering and cell based therapies where injury might occur when cells are harvested or handled.

### EXAMPLE 4

### Sheep Model for Therapeutic Effect of OP-1 after Intra-Articular Injection

This study will use N=12 adult female 1.5-2.5 year old crossbred sheep that are acclimatized for 14 days and pass a health status assessment before entry into the study. Under general anesthesia and using aseptic technique, all sheep will receive standardized 30 MPa impact injuries to both (left and right) medial femoral condyles by a 3 cm minimally invasive arthrotomy. Three weeks postoperatively the sheep will be sedated with diazepam (10 mg/kg) and ketamine (3-5 mg/kg) to allow aseptic preparation of knee for synoviocentesis and injection of test article, placebo or physiologic saline into the medial femorotibial joint according the to Table 3.

TABLE 3

| | | | | Week | | | | | |
|---|---|---|---|---|---|---|---|---|---|
| | | | | | 3 | 4 | | | |
| | | | # | 0 | intra-articular | | | | |
| Dose | Group | # animals | Knees | Surgery | injection | | 8 | 12 | 16 |
| two | Test-L | 9 | 9 | Impact | OP-1/P | OP-1/P | | | sacrifice |
| doses 3 | Placebo-R | | 9 | Injury | Placebo | Placebo | | | |
| & 4 | | | | | | | | | |
| weeks | | | | | | | | | |
| post | | | | | | | | | |
| injury | | | | | | | | | |
| saline | Saline | 3 | 6 | Impact | Saline | Saline | | | sacrifice |
| controls | control-R | | | Injury | | | | | |
| | Saline | | | | None | None | | | |
| | control-L | | | | | | | | |

| Total animals in | 12 | | | | | | |
|---|---|---|---|---|---|---|---|
| study | | | | | | | |
| Synovial Fluid | | x | x | x | x | x | x |
| Aspirate | | | | | | | |

All sheep will receive bilateral medial femoral condyle injuries. In the first group of nine sheep, one knee will receive the test article and the contralateral knee will receive a placebo consisting of the vehicle alone. Knee treatments will be allocated by a complete block design. A second group of three sheep will receive physiologic saline USP as a control for the effect of the placebo.

The study will follow the following procedure set forth in Table 4:

TABLE 4

| | |
|---|---|
| Day-14 to | Preconditioning, health maintenance program, foot |
| day-1 | trimming, Q-fever test |
| Week 0 | Surgery and impact injury to both knees of sheep. |
| Week 3, 4 | Synovial fluid collection. |
| | Synovial fluid harvested and OP-1 and placebo injected |
| | into respective joints. |
| Week 8, 12 | Synovial fluid harvested using aseptic technique and |
| | sedation. Freeze 2 aliquots synovial fluid (200 uL each) |
| | and process one fresh EDTA aliquot for total leukocyte |
| | count, differential counts and total protein determination. |
| Week 16 | Sacrifice all sheep. Harvest synovial fluid and tissues for |
| | detailed assessments |

### EXAMPLE 5

### Guinea Pig and Rabbit Models of Osteoarthritis

The Hartley guinea pig (spontaneous) and rabbit ACL-resection (induced) osteoarthritis models were utilized. Fourteen guinea pigs of either 3, 6 or 9 months of age were injected in the right knee with a phosphate buffered saline (PBS) solution containing 50 μg rhOP-1 at 3-week intervals for a period of 12 weeks. The left knee served as an untreated control.

In ten New Zealand White rabbits, the left ACL was resected and received either an injection into the joint of 100 μg rhOP-1 in a PBS solution or a control solution at 3-week intervals during a 12-week evaluation period. The right knee served as a non ACL-resected nontreated control in all animals.

All animals in both models were evaluated for gross appearance and histologic evidence of arthritic changes using a modified Mankin scale to grade the severity of degeneration. The untreated guinea pig knees developed a progression of arthritic changes from 3 to 6, 6 to 9 and 9 to 12 months of age with severe degeneration apparent grossly and histologically at 12 months of age. The OP-1 treatment had the most profound effect in preventing degeneration in the guinea pig at the early time periods. Gross and histologic degeneration in the knee at 9 months of age in rhOP-1 treated animals were similar to untreated animals at 6 months of age. At 12 months of age, the severity of degenerative changes was comparable. In the rabbit ACL-resected model OP-1 treatment showed slight improvement in the severity of degeneration in treated sites at the 12 week evaluation period. These results demonstrate that OP-1 has some beneficial effects in preventing or slowing early stage arthiritic changes.

### EXAMPLE 6

### Sheep Model of Meniscus Healing

A hole (6 mm diameter) and a longitudinal tear (2 cm long) sutured by non-resorbable thread were created in each medial meniscus of both knees of sheep. There were two treatment groups: OP-1 putty (3.5 mg OP-1/gram of Bovine type 1 collagen with carboxymethylcellulose) and a control group with no treatment other than the surgically created defect. The OP-1 treated animals received 0.3 mls (350 mcg) injected into the joint space just prior to closing the incision and then injected into the joint space 7 days after surgery.

6, 12 and 26 weeks after treatment, the animals will be euthanized. After euthanasia, the meniscus will be removed and cut in two parts, the anterior, longitudinal sutured tear and the posterior, with the hole. The sections will be stained with Masson's Trichrome and safranin O. Immunohistochemistry of the meniscus may also be performed using specific antibodies to detect collagen I, II, VI, S100, proteases MMP1.

A section of meniscus will be separated, embedded in OCT and frozen in liquid nitrogen. Sections obtained with a cryostat will be collected, homogenized and RNA prepared using Trizol reagent. RT-PCR will be performed to study gene expression of various markers including type I, type II, type II collagen and aggrecan as markers for extracellular matrix, TGF-β and IGF-2 as growth factors, MMP-1, MMP-3 and TIMP-1 as matrix degrading enzymes, and finally cyclin A, Bc1-2, BAX and caspase 3 as markers for proliferating and apoptotic state of cells. Other joint tissue will also be inspected and compared to controls for any gross differences which may be caused by OP-1.

Preliminary results on effect of OP-1 putty in holes in the avascular area of the meniscus indicate that in all the menisci with hole defects a positive effect was noted after treatment with OP-1 putty. The putty remained for the first six weeks, and later was reabsorbed and disappeared. Notably, there was considerable penetration of cells from the surface of the meniscus to the inside of the holes, which were mainly filled with fibrous tissue from the eighth week onwards.

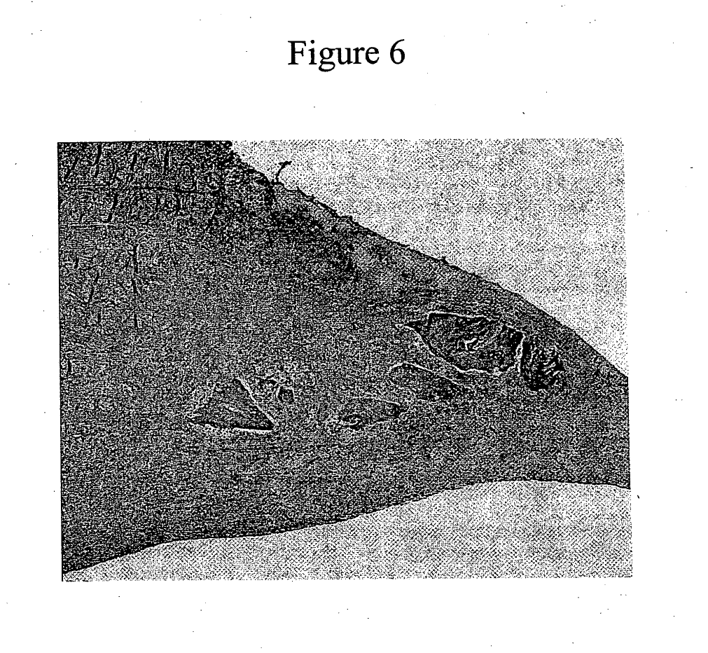

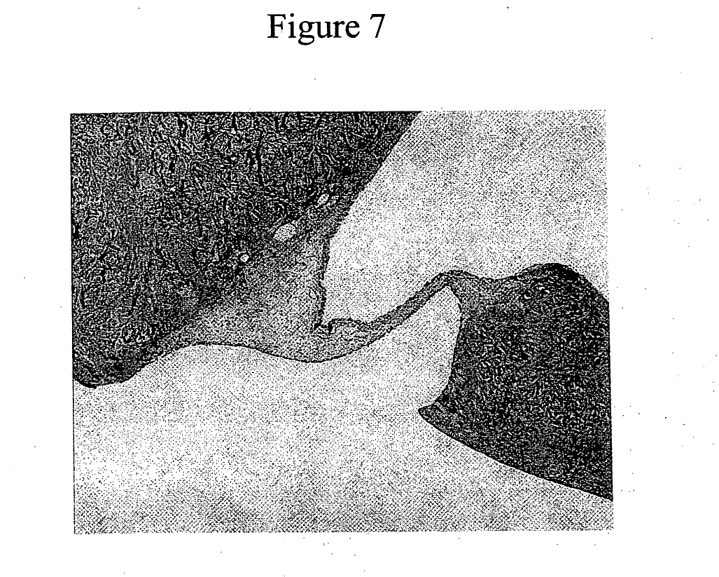

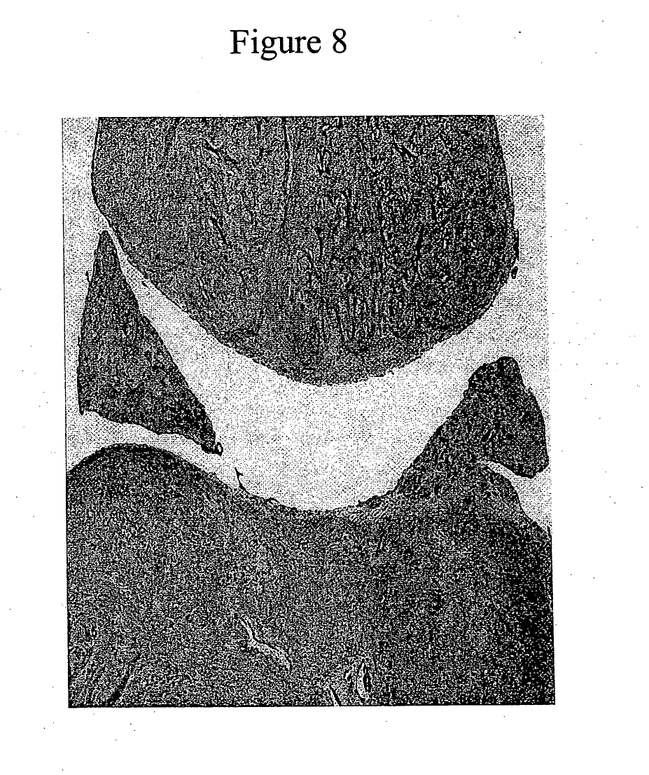

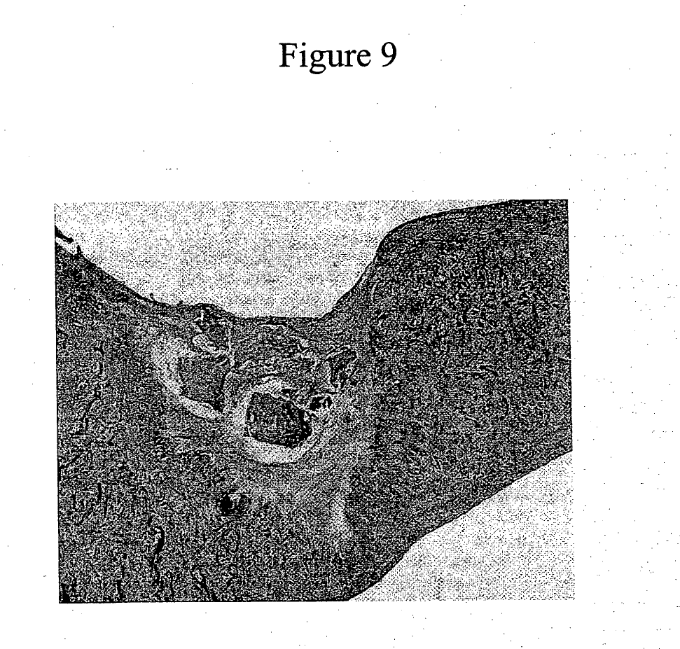

At 6 weeks, most of the control animals had little material filling the defects, and the material present was fibrous and whispy. In the OP-1 treated defects, there was more tissue present along with large particulate collagen. Cellular response appeared to be higher in the OP-1 group (see FIGS. 6 and 7). By 12 weeks, most of the control defects remained empty. Little cellular activity was seen along the periphery of the defect. The OP-1 treated defects still contained collagen particles, but there appeared to be an increase in cellularity surrounding the defect, and some progression to new tissue formation (see FIG. 8). After 25 weeks, fibrous bridging was seen in a few of the control animals, but most of this was tenuous in nature. The collagen particles disappeared from the defects in the OP-1 group and were replaced predominantly with fibrous tissue. Remodeling appeared to remain active (see FIG. 9).

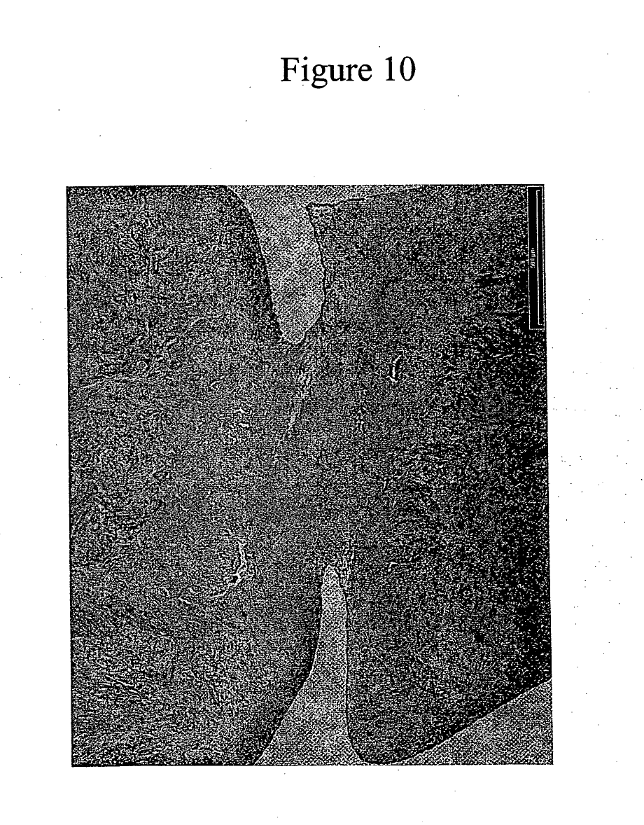

Preliminary results on the effect of OP-1 putty on the repair of menisci with longitudinal lesions were not conclusive. Only small differences were observed from the lesions treated with OP-1 when compared to the control group. This could be due to the fact that suturing does not provide adequate fixation, and that the protein does not integrate well because of the suture. In a few OP-1 treated animals bridging of the defect could be observed (see FIG. 10).

### EXAMPLE 7

### Sheep Model of Disc Repair and Regeneration

Experimental induction of controlled outer annular defects in sheep initiates a sequence of events which closely reproduces, pathologically and biochemically, the evolution of disc degeneration in man. Compositional changes include an alteration in the amount of, and the types of collagens synthesized by cells of the lesion site (Kaapa et al 1994a, b, 1995 Kaapa E. et al. (1995) Collagen synthesis and types I, III, IV, and VI collagens in an animal model of disc degeneration, Spine 20, 59-67; Kaapa E et al., (1994) Collagens in the injured porcine intervertebral disc, J. Orthop. Res. 12. 93-102; and Kaapa E et al., (1994) Proteoglycan chemistry in experimentally injured porcine intervertebral disk, J. Spin. Dis. 7, 296-306) loss of large high buoyant density aggrecan type proteoglycans and an elevation in levels of the small DS substituted proteoglycans decorin and biglycan in the injured disc (Melrose J. et al, (1992) A longitudinal study of the matrix changes induced in the intervertebral disc by surgical damage to the annulus fibrosus, J Orthop Res 10:665-676; Melrose J. et al., (1997) Topographical variation in the catabolism of aggrecan in an ovine annular lesion model of experimental disc degeneration J Spinal Disord 10:55-67; and Melrose J. et al., (1997) Elevated synthesis of biglycan and decorin in an ovine annular lesion model of experimental disc degeneration, Eur Spine J 6:376-84). Changes in the vascular supply to the cartilaginous end plate (CEP) (Moore R J et al., (1992) Changes in endplate vascularity after an outer anulus tear in the sheep, Spine 17:874-878) and remodelling of vertebral bone adjacent to experimental annular defects (Moore R J, et al. (1996) Remodeling of vertebral bone after outer anular injury in sheep, Spine 21:936-940.), changes in the biomechanical competence of “repaired” lesion affected discs (Latham J M et al., (1994) Mechanical consequences of annular tears and subsequent intervertebral disc degeneration, J Clin Biomech 9:211-9), and osteoarthritic changes in spinal facet joints (Moore R J et al., (1999) Osteoarthrosis of the facet joints resulting from anular rim lesions in sheep lumbar discs, Spine, 24:519-525) as a consequence of disc degeneration have also been noted.

A. The Ovine Annular Lesion Model

The sheep will be fasted for 24 h prior to surgery and anaesthesia will be induced with an intravenous injection of 1 g thiopentone. A lateral plain X-ray film will be taken to verify normal lumbar spine anatomy. General anaesthesia will be maintained after endotracheal intubation by 2.5% halothane and monitored by pulse oximetry and end tidal CO₂ measurement. The left flank from the ribs to the iliac crest will be prepared for sterile surgery. The sheep will receive an intramuscular injection of 1200 mg penicillin. A skin incision will be made on the left side immediately anterior to the transverse processes of the spine and the lumbar spine will be exposed by blunt dissection using an anterior muscle-splitting technique. The vascular and neural anatomy will be respected and bleeding will be controlled by direct pressure or electrocautery as required.

A total of twelve two year old sheep will receive controlled annular lesions in their L1-L2, L3-L4 and L5-L6 discs by incision through the left anterolateral annulus fibrosus parallel and adjacent to the cranial endplate using a #11 scalpel blade to create a lesion measuring 4 mm long×5 mm deep. The intervening lumbar discs (L2-L3, L4-L5) will not be incised.

The incised discs will receive one of 3 therapies, (I) no treatment, (II) lactose solution or (III) lactose containing OP-1. In all sheep the L3-L4 disc will receive an annular lesion with no treatment. In 4 sheep the L1-L2 discs will be treated with lactose solution only and the L5-L6 disc will be treated with lactose plus OP-1. In the remaining 4 sheep the treatments in the L1-L2 and L5-L6 discs will be reversed to avoid any potential outcome bias associated with spinal level. A non-operated disc must remain between treated discs to allow for adequate anchorage of FSUs in subsequent mechanical testing (see below). A wire suture will be used to identify the craniad operated level for later identification purposes both in X-rays and for morphological identification. Three additional non-operated animals will also be used as controls for the biomechanical study.

Degeneration following annular incision is well established in the sheep (Osti O L et al., (1990) Volvo Award for Basic Science, Annulus tears and intervertebral disc degeneration. An experimental study using an animal model, Spine 15:762-7) and can be expected to show the earliest radiographic and histochemical evidence after 12 weeks. Three months after induction of the annular lesions the sheep will be killed by intravenous injection of 6.5 g sodium pentobarbitone and the lumbar spines will be radiographed to evaluate disc calcification, excised and processed for biomechanical (n=8) and histochemical (n=4) analyses, and, after the biomechanical testing the same discs will be zonally dissected for compositional analyses.

B. Compositional Analysis of Disc Tissues

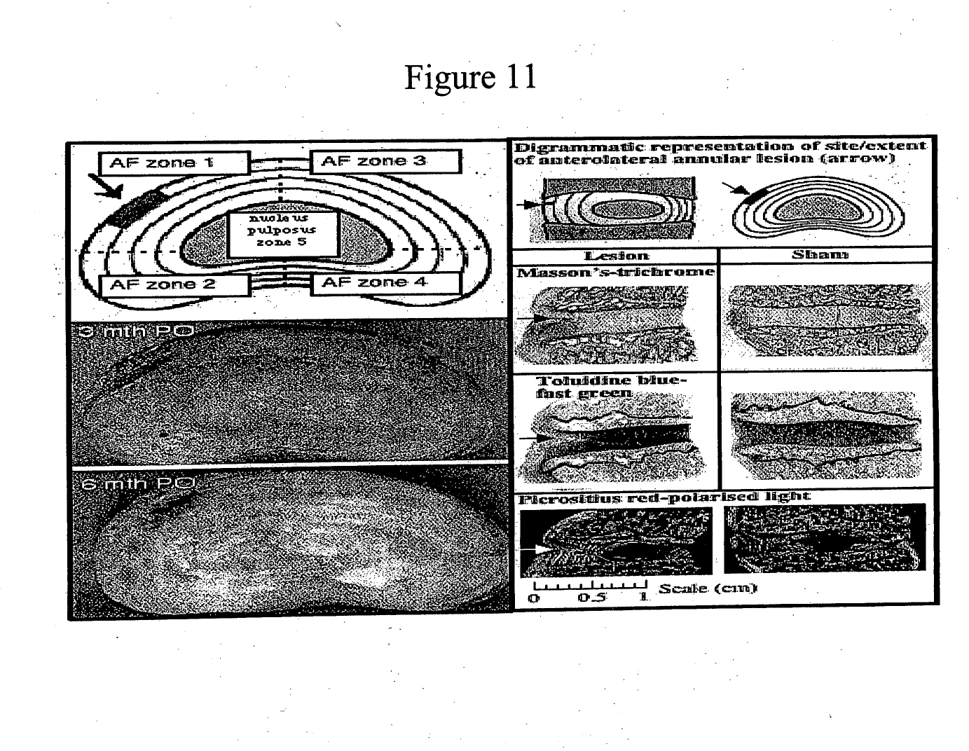

Intervertebral disc tissues will be zonally dissected into annular quadrants and nucleus pulposus as depicted in FIG. 11.

C. Determination of Proteoglycan and Collagen Contents of Disc Tissues

Samples of annulus fibrosus and nucleus pulposus will be finely diced over ice and representative portions of each tissue zone of known wet weight will be freeze dried to constant weight. The difference between the starting and final weights of the tissues will provide their water contents. Triplicate portions (1-2 mg) of the dried tissues will be hydrolyzed in 6M HCl at 110° C. for 16 h and aliquots of the neutralized digests assayed for hydroxyproline as a measure of the tissue collagen content (Melrose J et al., (1992) A longitudinal study of the matrix changes induced in the intervertebral disc by surgical damage to the annulus fibrosus, J Orthop Res 10:665-676; Melrose J et al., (1994a) Proteoglycan heterogeneity in the normal adult ovine intervertebral disc, Matrix 14:61-75; Melrose J et al., (1994b) Variation in the composition of the ovine intervertebral disc with spinal level and in its constituent proteoglycans, Vet Comp Orthop Traum 7:70-76; Melrose J et al., (1991) The influence of scoliosis and ageing on proteoglycan heterogeneity in the human intervertebral disc J Orthop Res 9:68-77; and Melrose J et al., (1996) Intervertebral disc reconstitution after chemonucleolysis with chymopapain is dependent on dosage: an experimental study in beagle dogs Spine 21:9-17). Triplicate portions of dried tissues (˜2 mg) will also be digested with papain and aliquots of the solubilized tissue assayed for sulphated glycosaminoglycan using the metachromatic dye 1,9-dimethylmethylene blue as a measure of tissue proteoglycan (see Melrose et al 1991, 1992, 1994, 1996, supra).

D. Histochemical and Immunohistochemical Analyses

Spinal motion segments that are designated for histochemical analysis will be isolated by cutting through the cranial and caudal vertebral bodies close to the cartilaginous endplates using a bone saw. Entire disc specimens including the adjacent vertebral body segments will be fixed en bloc in either 10% neutral buffered formalin or Histochoice® for 56 h and decalcified in several changes of 10% formic acid in 5% NBF for 2 weeks with constant agitation until complete decalcification is confirmed using a Faxitron HP43855A X-ray cabinet (Hewlett Packard, McMinnville, USA).

Sagittal slices (5 mm thick) of the decalcified disc-vertebral body specimens will be dehydrated through graded ethanol solutions by standard histological methods and embedded in paraffin wax. Paraffin sections 4 μm thick will be prepared for histochemical staining and mounted on Superfrost Plus glass microscope slides (Menzel-Glaser) and dried at 85° C. for 30 min then at 55° C. overnight. The sections will be deparaffinized in xylene (4 changes×2 min) and rehydrated through graded ethanol washes (100-70% v/v) to water.

Three sections from all blocks will be stained with haematoxylin and eosin. These sections will be coded and examined by an independent histopathologist who will compare the histological characteristics of those levels that received annular incision only with those that were incised and received rhOP-1. A four-point semi-quantitative grading system will be used to assess the microscopic features. Collagen architecture will also be examined in sections stained with Masson's trichrome and picro-sirius red using polarized light microscopy.

The immunohistochemistry procedures will be performed using a Sequenza cassette and disposable Coverplate immunostaining system as described earlier (Melrose J et al., (2002) Perlecan, the Multi-domain Proteoglycan of Basement Membrane is also a Prominent Pericellular Component of Hypertrophic Chondrocytes of Ovine Vertebral Growth Plate and Cartilaginous End Plate Cartilage, Histochem. Cell Biol. 118, 269-280; Melrose J et al., (2002) Increased nerve and blood-vessel in-growth associated with proteoglycan depletion in an ovine annular lesion model of experimental disc degeneration, Spine 27, 1278-85; Melrose J et al., (2002) Comparison of the morphology and growth characteristics of intervertebral disc cells, synovial fibroblasts and articular chondrocytes in monolayer and alginate bead cultures, Eur. Spine J. 12, 57-65; Melrose J et al. (2001) Differential expression of proteoglycan epitopes and growth characteristics of ovine intervertebral disc cells grown in alginate beads, Cells Tissues Organs 168:137-146; Melrose J et al., (2003) Perlecan, the multi domain HS-proteoglycan of basement membranes is a prominent extracellular and pericellular component of the cartilaginous vertebral body rudiments, vertebral growth plates and intervertebral discs of the developing human spinal column, J Histochem Cytochem 51:1331-1341; Melrose J et al., (2000) Differential Expression of Proteoglycan epitopes by ovine intervertebral disc cells grown in alginate bead culture, J. Anat. 197:189-198; Melrose J et al., (2002) Spatial and Temporal Localisation of Transforming Growth Factor-β, Fibroblast Growth Factor-2, Osteonectin and Identification of Cells Expressing α-Smooth Muscle Actin in the Injured Annulus Fibrosus: Implications for Extracellular Matrix Repair, Spine 27:1756-1764; and Knox S et al., (2002) Not all perlecans are created equal: interactions with fibroblast growth factor-2 (FGF-2) and FGF receptors, J. Biol. Chem. 277:14657-14665). Endogenous peroxidase activity will be initially blocked by incubating the tissue sections with 3% H₂O₂. This will be followed by pre-digestion of the tissue sections with combinations of chondroitinase ABC (0.25 U/ml) in 20 mM Tris-acetate buffer pH 8.0 for 1 h at 37° C., bovine testicular hyaluronidase 1000 U/ml for 1 h at 37° C. in phosphate buffer pH 5.0, followed by three washes in 20 mM Tris-HCl pH 7.2 0.5M NaCl (TBS) or proteinase-K (DAKO S3020) for 6 min at room temperature to expose antigenic epitopes. The tissues will then be blocked for 1 h in 20% normal swine serum and be probed with a number of primary antibodies to large and small proteoglycans and collagens (Table 5). Negative control sections will also be processed either omitting primary antibody or substituting an irrelevant isotype matched primary antibody for the authentic primary antibody of interest. Horseradish peroxidase or alkaline phosphatase conjugated secondary antibodies will be used for detection using 0.05% 3,3′-diaminobenzidene dihydrochloride and 0.03% H₂O₂ in TBS or Nova RED substrates. The stained slides will be examined by bright-field microscopy and photographed using a Leica MPS 60 photomicroscope digital camera system.

TABLE 5
Primary antibodies to proteoglycan and collagen core protein epitopes

| Primary Antibody epitope | Clone (isotype) |
|---|---|
| Large Proteoglycans | |
| Aggrecan | AD 11-2A9 (IgG) |
| Perlecan | A76 (IgG₁) |
| Versican | A1S1D1D1 (IgG) |
| Small proteoglycans | |
| Decorin | 6-B-6 (IgG) |
| Biglycan | LF-96 (rabbit IgG) |
| Fibromodulin | Rabbit polyclonal |
| Collagen | |
| Type I | I8H5 (IgG₁) |
| Type II | II-4CII (IgG₁) |
| Type IV | CIV-22 (IgG₁) |
| Type VI | Rabbit polyclonal |
| Type X | Mouse polyclonal |

E. Biomechanical Assessment of Spinal Motion Segments

Non-destructive biomechanical range of motion (ROM) analysis will be conducted on each functional spinal unit (FSU) in various planes of motion (flexion-extension, lateral bending, compression and torsion). Each FSU comprises two adjacent vertebrae, the intervening disc and associated ligaments.

A specially designed jig, based on that developed by Callaghan and McGill, allows pure torsion and bending moments to be applied to each FSU while maintaining a constant axial load. This combined loading is a close simulation of the physiological loads experienced by the spine in-vivo.

Four FSUs will be tested: non-operated control levels; levels that were incised; levels that were incised and treated with OP-1 and carrier and levels that were incised and treated with carrier alone. Each FSU will be mounted in two aluminum alloy cups and secured with cold cure dental cement. Care will be taken to ensure that the intervertebral disc is aligned with the cups. Prior to the commencement of testing each FSU will be preloaded to a stress of 0.5 MPa until a reproducible state of hydration is achieved. This is used as the baseline prior to each test. The preload stress of 0.5 MPa simulates relaxed standing and is based on in-vivo measurement of intradiscal pressure (Wilke H-J et al., (1999) New in vivo measurements of pressures in the intervertebral disc in daily life, Spine 24:755-62). A ±5 Nm torsional load and ±1 Nm flexion-extension, lateral bending load will be applied over 10 cycles whilst under a constant 0.5 MPa axial load. A cyclic axial load (0-1000N over 10 cycles) will be applied to investigate the axial compression response of the IVD.

F. Pilot Studies

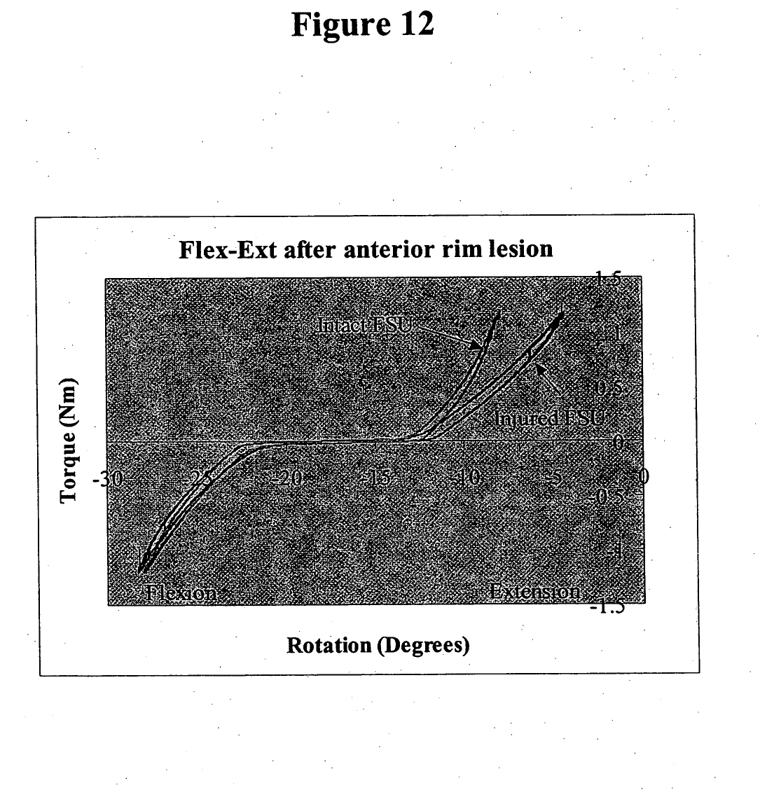

Pilot studies have been completed on both sheep and kangaroo spines to verify the experimental techniques. FIG. 12 demonstrates typical ‘Torque versus Rotation’ plots of a sheep FSU over 10 flexion-extension loading cycles. The two plots represent the FSU before and after a circumferential anterior annular rim lesion. It can be seen that the annular cut resulted in increased range of motion (ROM) during extension, whilst flexion ROM was unaffected. This increased ROM overall represents an increase in spinal instability. Another observation is the high repeatability of the loading cycles, which verifies the reproducibility of the testing setup.

Data analysis will include stiffness in the linear region during the fifth loading cycle, hysteresis and strain energy and the extent of the neutral zone. Data from the non-operative levels will be compared with incised levels with and without OP-1 and a one-way repeated measures analysis of variance will be conducted on each of the biomechanical parameters.

### EXAMPLE 8

### The Effect Of OP-1 On Chondral And Microfracture Treated Cartilage Defects In A Goat Model

This study will evaluate the effects of OP-1 on the amount and composition of the reparative tissue induced by a microfracture procedure in a goat model. A total of 24 adult male goats (ages 1.5 to 3 years) weighing approximately 25 kg will be used. Prior to surgery, the knee joints will be roentgenographically examined to exclude animals with degenerative joint disease or other noted orthopedic problems. One 8 mm (on a side) square chondral defect (cartilage removed down to tidemark-the calcified cartilage layer) will be produced in the trochlear groove of the right knees (stifle joints) of all animals. In 12 of the goats this chondral defect will serve as the site to the treated (Groups IA and IB (see table 4 below). The right knee joints of 12 of the animals will then undergo microfracture treatment (Groups IIA and IIB). 16 microfracture holes will be produced using a pick of approximately 1 mm diameter.

Immediately postoperative, approximately 0.3 ml of OP-1 putty (collagen +CMC) hydrated with saline will be injected into the synovial fluid of the joint. At seven days, a second injection will be administered. In 6 of the animals in the chondral defect group (IB) and in 6 of the animals in the microfracture group (IIB) only vehicle will be delivered.

TABLE 6

| | Type of | Treatment (+ or − | |
|---|---|---|---|
| Group | Lesion | OP-1) | Sample Size |
| IA | Chondral | + | 6 |
| IB | Chondral | − | 6 |
| IIA | Microfracture | + | 6 |
| IIB | Microfracture | − | 6 |

All animals will be sacrificed 16 weeks after surgery. All of the sites will be prepared for histomorphometric evaluation. One histological section from the center portion of each defect will be evaluated. The total area and the percentages of specific tissue types (articular cartilage, hyaline cartilage, fibrocartilage and fibrous tissue) filling the original chondral defect region will be determined using a grid in the eyepiece of the microscope. Well accepted histological criteria for tissue types will be employed (see, e.g., Wang Q., et al. Healing of defects in canine articular cartilage: distribution of nonvascular alpha smooth muscle actin-containing cells, Wound Repair Regen. 8, pp. 145-158 (2000); Breinan H A, et al., Healing of canine articular cartilage defects treated with microfracture, a type II collagen matrix, or cultured autologous chondrocytes, J. Orthop. Res. 18, pp. 781-789 (2000); and Breinan, H A, et al., Effect of cultured autologous chondrocytes on repair of chondral defects in a canine model, J. Bone Joint Surg. 79A, pp. 1439-1451 (1997)).

We claim:

1. A method of repairing a cartilage defect in a patient comprising the step of administering into the cartilage or into the area surrounding the cartilage a composition comprising a therapeutically effective amount of a morphogenic protein.

2. The method of claim 1, wherein the cartilage is selected from the group consisting of articular cartilage and non-articular cartilage.

3. The method of claim 2, wherein the non-articular cartilage is selected from the group consisting of a meniscus and an intervertebral disc.

4. The method of claim 1, wherein the area surrounding the cartilage is synovial fluid.

5. The method of claim 1, wherein the morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and amino acid sequence variants thereof.

6. The method of claim 1, wherein the morphogenic protein comprises an amino acid sequence having at least 70% homology with the C-terminal 102-106 amino acids, including the conserved seven cysteine domain, of human OP-1, said morphogenic protein being capable of inducing repair of the cartilage defect.

7. The method of claim 1, wherein the morphogenic protein is selected from the group consisting of OP-1, BMP-5, BMP-6, GDF-5, GDF-6, GDF-7, CDMP-1, CDMP-2 and CDMP-3.

8. The method of claim 7, wherein said morphogenic protein is OP-1.

9. The method of claim 1, wherein the composition is selected from the group consisting of a gel, an aqueous solution, a paste and a putty.

10. The method of claim 9, wherein the composition is formulated as a sustained release formulation or as a delayed clearance formulation.

11. The method of claim 9, wherein the composition is an injectable formulation.

12. The method of claim 9, wherein the composition is a gel.

13. The method of claim 9, wherein the composition is an aqueous solution.

14. The method of claim 10, wherein the composition comprises polyethylene glycol.

15. The method of claim 10, wherein the morphogenic protein is glycosylated.

16. A method of regenerating or producing cartilage in a patient comprising the step of administering into the cartilage or the area surrounding the cartilage a composition comprising a therapeutically effective amount of a morphogenic protein.

17. The method of claim 16, wherein the cartilage is selected from the group consisting of articular cartilage and non-articular cartilage.

18. The method of claim 17, wherein the non-articular cartilage is selected from the group consisting of a meniscus and an intervertebral disc.

19. The method of claim 16, wherein the area surrounding the cartilage is synovial fluid.

20. The method of claim 16, wherein the morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and amino acid sequence variants thereof.

21. The method of claim 16, wherein the morphogenic protein comprises an amino acid sequence having at least 70% homology with the C-terminal 102-106 amino acids, including the conserved seven cysteine domain, of human OP-1, said morphogenic protein being capable of inducing repair of the cartilage defect.

22. The method of claim 20, wherein the morphogenic protein is selected from the group consisting of OP-1, BMP-5, BMP-6, GDF-5, GDF-6, GDF-7, CDMP-1, CDMP-2 and CDMP-3.

23. The method of claim 20, wherein said morphogenic protein is OP-1.

24. The method of claim 16, wherein the composition is selected from the group consisting of a gel, an aqueous solution, a paste and a putty.

25. The method of claim 24, wherein the composition is formulated as a sustained release formulation or as a delayed clearance formulation.

26. The method of claim 24, wherein the composition is an injectable formulation.

27. The method of claim 24, wherein the composition is a gel.

28. The method of claim 24, wherein the composition is an aqueous solution.

29. The method of claim 25, wherein the composition comprises polyethylene glycol.

30. The method of claim 25, wherein the morphogenic protein is glycosylated.

31. A method of promoting cartilage growth or accelerating cartilage formation in a patient comprising the step of administering into the cartilage or into the area surrounding the cartilage a composition comprising a therapeutically effective amount of a morphogenic protein.

32. The method of claim 31, wherein the cartilage is selected from the group consisting of articular cartilage and non-articular cartilage.

33. The method of claim 32, wherein the non-articular cartilage is selected from the group consisting of a meniscus and an intervertebral disc.

34. The method of claim 31, wherein the area surrounding the cartilage is synovial fluid.

35. The method of claim 31, wherein the morphogenic protein is selected from the group consisting of OP-1, OP-2, OP-3, BMP-2, BMP-3, BMP-4, BMP-5, BMP-6, BMP-8, BMP-9, BMP-10, BMP-1 1, BMP-12, BMP-13, BMP-15, BMP-16, BMP-17, BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and amino acid sequence variants thereof.

36. The method of claim 31, wherein the morphogenic protein comprises an amino acid sequence having at least 70% homology with the C-terminal 102-106 amino acids, including the conserved seven cysteine domain, of human OP-1, said morphogenic protein being capable of inducing repair of the cartilage defect.

37. The method of claim 35, wherein the morphogenic protein is selected from the group consisting of OP-1, BMP-5, BMP-6, GDF-5, GDF-6, GDF-7, CDMP-1, CDMP-2 and CDMP-3.

38. The method of claim 37, wherein said morphogenic protein is OP-1.

39. The method of claim 31, wherein the composition is selected from the group consisting of a gel, an aqueous solution, a paste and a putty.

40. The method of claim 39, wherein the composition is formulated as a sustained release formulation or as a delayed clearance formulation.

41. The method of claim 39, wherein the composition is an injectable formulation.

42. The method of claim 39, wherein the composition is a gel.

43. The method of claim 39, wherein the composition is an aqueous solution.

44. The method of claim 40, wherein the composition comprises polyethylene glycol.

45. The method of claim 40, wherein the morphogenic protein is glycosylated.

46. A method of preventing cartilage degradation or treating cartilage injury or degenerative disease or disorder in a patient comprising the step of administering into the cartilage or into the area surrounding the cartilage a composition comprising a therapeutically effective amount of a morphogenic protein.

47. The method of claim 46, wherein the cartilage is selected from the group consisting of articular cartilage and non-articular cartilage.

48. The method of claim 47, wherein the non-articular cartilage is selected from the group consisting of a meniscus and an intervertebral disc.

49. The method of claim 46, wherein the area surrounding the cartilage is synovial fluid.

50. The method of claim 46, wherein the morphogenic protein is selected from the group consisting of OP-1, OP-2, OP-3, BMP-2, BMP-3, BMP-4, BMP-5, BMP-6, BMP-8, BMP-9, BMP-10, BMP-11, BMP-12, BMP-13, BMP-15, BMP-16, BMP-17. BMP-18, DPP, Vg1, Vgr, 60A protein, GDF-1, GDF-2, GDF-3, GDF-5, GDF-6, GDF-7, GDF-8, GDF-9, GDF-10, GDF-11, GDF-12, CDMP-1, CDMP-2, CDMP-3, NODAL, UNIVIN, SCREW, ADMP, NEURAL, and amino acid sequence variants thereof.

51. The method of claim 46, wherein the morphogenic protein comprises an amino acid sequence having at least 70% homology with the C-terminal 102-106 amino acids, including the conserved seven cysteine domain, of human OP-1, said morphogenic protein being capable of inducing repair of the cartilage defect.

52. The method of claim 51, wherein the morphogenic protein is selected from the group consisting of OP-1, GDF-5, GDF-6, GDF-7, CDMP-1, CDMP-2 and CDMP-3.

53. The method of claim 52, wherein said morphogenic protein is OP-1.

54. The method of claim 46, wherein the composition is selected from the group consisting of a gel, an aqueous solution, a paste and a putty.

55. The method of claim 54, wherein the composition is formulated as a sustained release formulation or as a delayed clearance formulation.

56. The method of claim 54, wherein the composition is an injectable formulation.

57. The method of claim 54, wherein the composition is a gel.

58. The method of claim 54, wherein the composition is an aqueous solution.

59. The method of claim 55, wherein the composition comprises polyethylene glycol.

60. The method of claim 55, wherein the morphogenic protein is glycosylated.

61. The method of claim 46, wherein the tissue injury or degenerative disease is selected from the group consisting of osteoarthritis, meniscus tears, ACL injury and disc degeneration.
